SEQUENCE LISTING (14 sequences)

SEQ ID NO: 1 - protein, 566 aa, Homo sapiens
MERAAPSRRVPLPLLLLGGLALLAAGVDADVLLEACCADGHRMATHQKDCSLPYATESKECRMVQEQCCHSQLEELHCATGISLANEQDRCATPHGDNASLEATFVKRCCHCCLLGRAAQAQGQSCEYSLMVGYQCGQVFRACCVKSQETGDLDVGGLQETDKIIEVEEEQEDPYLNDRCRGGGPCKQQCRDTGDEVVCSCFVGYQLLSDGVSCEDVNECITGSHSCRLGESCINTVGSFRCQRDSSCGTGYELTEDNSCKDIDECESGIHNCLPDFICQNTLGSFRCRPKLQCKSGFIQDALGNCIDINECLSISAPCPIGHTCINTEGSYTCQKNVPNCGRGYHLNEEGTRCVDVDECAPPAEPCGKGHRCVNSPGSFRCECKTGYYFDGISRMCVDVNECQRYPGRLCGHKCENTLGSYLCSCSVGFRLSVDGRSCEDINECSSSPCSQECANVYGSYQCYCRRGYQLSDVDGVTCEDIDECALPTGGHICSYRCINIPGSFQCSCPSSGYRLAPNGRNCQDIDECVTGIHNCSINETCFNIQGGFRCLAFECPENYRRSAAT

SEQ ID NO: 2 - DNA, 1701 bp, Homo sapiens
atggagcgcgccgcgccgtcgcgccgggtcccgcttccgctgctgctgctcggcggccttgcgctgctggcggccggagtggacgcggatgtcctcctggaggcctgctgtgcggacggacaccggatggccactcatcagaaggactgctcgctgccatatgctacggaatccaaagaatgcaggatggtgcaggagcagtgctgccacagccagctggaggagctgcactgtgccacgggcatcagcctggccaacgagcaggaccgctgtgccacgccccacggtgacaacgccagcctggaggccacatttgtgaagaggtgctgccattgctgtctgctggggagggcggcccaggcccagggccagagctgcgagtacagcctcatggttggctaccagtgtggacaggtcttccgggcatgctgtgtcaagagccaggagaccggagatttggatgtcgggggcctccaagaaacggataagatcattgaggttgaggaggaacaagaggacccatatctgaatgaccgctgccgaggaggcgggccctgcaagcagcagtgccgagacacgggtgacgaggtggtctgctcctgcttcgtgggctaccagctgctgtctgatggtgtctcctgtgaagatgtcaatgaatgcatcacgggcagccacagctgccggcttggagaatcctgcatcaacacagtgggctctttccgctgccagcgggacagcagctgcgggactggctatgagctcacagaggacaatagctgcaaagatattgacgagtgtgagagtggtattcataactgcctccccgattttatctgtcagaatactctgggatccttccgctgccgacccaagctacagtgcaagagtggctttatacaagatgctctaggcaactgtattgatatcaatgagtgtttgagtatcagtgccccgtgccctatcgggcatacatgcatcaacacagagggctcctacacgtgccagaagaacgtgcccaactgtggccgtggctaccatctcaacgaggagggaacgcgctgtgttgatgtggacgagtgcgcgccacctgctgagccctgtgggaagggacatcgctgcgtgaactctcccggcagtttccgctgcgaatgcaagacgggttactattttgacggcatcagcaggatgtgtgtcgatgtcaacgagtgccagcgctaccccgggcgcctgtgtggccacaagtgcgagaacacgctgggctcctacctctgcagctgttccgtgggcttccggctctctgtggatggcaggtcatgtgaagacatcaatgagtgcagcagcagcccctgtagccaggagtgtgccaacgtctacggctcctaccagtgttactgccggcgaggctaccagctcagcgatgtggatggagtcacctgtgaagacatcgacgagtgcgccctgcccaccgggggccacatctgctcctaccgctgcatcaacatccctggaagcttccagtgcagctgcccctcgtctggctacaggctggcccccaatggccgcaactgccaagacattgatgagtgtgtgactggcatccacaactgctccatcaacgagacctgcttcaacatccagggcggcttccgctgcctggccttcgagtgccctgagaactaccgccgctccgcagccacatga

SEQ ID NO: 3 - protein, 601 aa, Homo sapiens
MERAAPSRRVPLPLLLLGGLALLAAGVDADVLLEACCADGHRMATHQKDCSLPYATESKECRMVQEQCCHSQLEELHCATGISLANEQDRCATPHGDNASLEATFVKRCCHCCLLGRAAQAQGQSCEYSLMVGYQCGQVFRACCVKSQETGDLDVGGLQETDKIIEVEEEQEDPYLNDRCRGGGPCKQQCRDTGDEVVCSCFVGYQLLSDGVSCEDVNECITGSHSCRLGESCINTVGSFRCQRDSSCGTGYELTEDNSCKDIDECESGIHNCLPDFICQNTLGSFRCRPKLQCKSGFIQDALGNCIDINECLSISAPCPIGHTCINTEGSYTCQKNVPNCGRGYHLNEEGTRCVDVDECAPPAEPCGKGHRCVNSPGSFRCECKTGYYFDGISRMCVDVNECQRYPGRLCGHKCENTLGSYLCSCSVGFRLSVDGRSCEDINECSSSPCSQECANVYGSYQCYCRRGYQLSDVDGVTCEDIDECALPTGGHICSYRCINIPGSFQCSCPSSGYRLAPNGRNCQDIDECVTGIHNCSINETCFNIQGGFRCLAFECPENYRRSAATQKSKKGRQNTPAGSSKEDCRVLPWKQGLEDTHLDA

SEQ ID NO: 4 - DNA, 1806 bp, Homo sapiens
atggagcgcgccgcgccgtcgcgccgggtcccgcttccgctgctgctgctcggcggccttgcgctgctggcggccggagtggacgcggatgtcctcctggaggcctgctgtgcggacggacaccggatggccactcatcagaaggactgctcgctgccatatgctacggaatccaaagaatgcaggatggtgcaggagcagtgctgccacagccagctggaggagctgcactgtgccacgggcatcagcctggccaacgagcaggaccgctgtgccacgccccacggtgacaacgccagcctggaggccacatttgtgaagaggtgctgccattgctgtctgctggggagggcggcccaggcccagggccagagctgcgagtacagcctcatggttggctaccagtgtggacaggtcttccgggcatgctgtgtcaagagccaggagaccggagatttggatgtcgggggcctccaagaaacggataagatcattgaggttgaggaggaacaagaggacccatatctgaatgaccgctgccgaggaggcgggccctgcaagcagcagtgccgagacacgggtgacgaggtggtctgctcctgcttcgtgggctaccagctgctgtctgatggtgtctcctgtgaagatgtcaatgaatgcatcacgggcagccacagctgccggcttggagaatcctgcatcaacacagtgggctctttccgctgccagcgggacagcagctgcgggactggctatgagctcacagaggacaatagctgcaaagatattgacgagtgtgagagtggtattcataactgcctccccgattttatctgtcagaatactctgggatccttccgctgccgacccaagctacagtgcaagagtggctttatacaagatgctctaggcaactgtattgatatcaatgagtgtttgagtatcagtgccccgtgccctatcgggcatacatgcatcaacacagagggctcctacacgtgccagaagaacgtgcccaactgtggccgtggctaccatctcaacgaggagggaacgcgctgtgttgatgtggacgagtgcgcgccacctgctgagccctgtgggaagggacatcgctgcgtgaactctcccggcagtttccgctgcgaatgcaagacgggttactattttgacggcatcagcaggatgtgtgtcgatgtcaacgagtgccagcgctaccccgggcgcctgtgtggccacaagtgcgagaacacgctgggctcctacctctgcagctgttccgtgggcttccggctctctgtggatggcaggtcatgtgaagacatcaatgagtgcagcagcagcccctgtagccaggagtgtgccaacgtctacggctcctaccagtgttactgccggcgaggctaccagctcagcgatgtggatggagtcacctgtgaagacatcgacgagtgcgccctgcccaccgggggccacatctgctcctaccgctgcatcaacatccctggaagcttccagtgcagctgcccctcgtctggctacaggctggcccccaatggccgcaactgccaagacattgatgagtgtgtgactggcatccacaactgctccatcaacgagacctgcttcaacatccagggcggcttccgctgcctggccttcgagtgccctgagaactaccgccgctccgcagccacgcagaaatccaagaagggaaggcagaacaccccagcgggatcaagtaaagaggactgcagggttcttccatggaagcaggggttggaggatacccaccttgatgcctag

SEQ ID NO: 5 - protein, 683 aa, Homo sapiens
MERAAPSRRVPLPLLLLGGLALLAAGVDADVLLEACCADGHRMATHQKDCSLPYATESKECRMVQEQCCHSQLEELHCATGISLANEQDRCATPHGDNASLEATFVKRCCHCCLLGRAAQAQGQSCEYSLMVGYQCGQVFRACCVKSQETGDLDVGGLQETDKIIEVEEEQEDPYLNDRCRGGGPCKQQCRDTGDEVVCSCFVGYQLLSDGVSCEDVNECITGSHSCRLGESCINTVGSFRCQRDSSCGTGYELTEDNSCKDIDECESGIHNCLPDFICQNTLGSFRCRPKLQCKSGFIQDALGNCIDINECLSISAPCPIGHTCINTEGSYTCQKNVPNCGRGYHLNEEGTRCVDVDECAPPAEPCGKGHRCVNSPGSFRCECKTGYYFDGISRMCVDVNECQRYPGRLCGHKCENTLGSYLCSCSVGFRLSVDGRSCEDINECSSSPCSQECANVYGSYQCYCRRGYQLSDVDGVTCEDIDECALPTGGHICSYRCINIPGSFQCSCPSSGYRLAPNGRNCQDIDECVTGIHNCSINETCFNIQGGFRCLAFECPENYRRSAATRCERLPCHENRECSKLPLRITYYHLSFPTNIQAPAVVFRMGPSSAVPGDSMQLAITGGNEEGFFTTRKVSPHSGVVALTKPVPEPRDLLLTVKMDLSRHGTVSSFVAKLFIFVSAEL

SEQ ID NO: 6 - DNA, 2052 bp, Homo sapiens
atggagcgcgccgcgccgtcgcgccgggtcccgcttccgctgctgctgctcggcggccttgcgctgctggcggccggagtggacgcggatgtcctcctggaggcctgctgtgcggacggacaccggatggccactcatcagaaggactgctcgctgccatatgctacggaatccaaagaatgcaggatggtgcaggagcagtgctgccacagccagctggaggagctgcactgtgccacgggcatcagcctggccaacgagcaggaccgctgtgccacgccccacggtgacaacgccagcctggaggccacatttgtgaagaggtgctgccattgctgtctgctggggagggcggcccaggcccagggccagagctgcgagtacagcctcatggttggctaccagtgtggacaggtcttccgggcatgctgtgtcaagagccaggagaccggagatttggatgtcgggggcctccaagaaacggataagatcattgaggttgaggaggaacaagaggacccatatctgaatgaccgctgccgaggaggcgggccctgcaagcagcagtgccgagacacgggtgacgaggtggtctgctcctgcttcgtgggctaccagctgctgtctgatggtgtctcctgtgaagatgtcaatgaatgcatcacgggcagccacagctgccggcttggagaatcctgcatcaacacagtgggctctttccgctgccagcgggacagcagctgcgggactggctatgagctcacagaggacaatagctgcaaagatattgacgagtgtgagagtggtattcataactgcctccccgattttatctgtcagaatactctgggatccttccgctgccgacccaagctacagtgcaagagtggctttatacaagatgctctaggcaactgtattgatatcaatgagtgtttgagtatcagtgccccgtgccctatcgggcatacatgcatcaacacagagggctcctacacgtgccagaagaacgtgcccaactgtggccgtggctaccatctcaacgaggagggaacgcgctgtgttgatgtggacgagtgcgcgccacctgctgagccctgtgggaagggacatcgctgcgtgaactctcccggcagtttccgctgcgaatgcaagacgggttactattttgacggcatcagcaggatgtgtgtcgatgtcaacgagtgccagcgctaccccgggcgcctgtgtggccacaagtgcgagaacacgctgggctcctacctctgcagctgttccgtgggcttccggctctctgtggatggcaggtcatgtgaagacatcaatgagtgcagcagcagcccctgtagccaggagtgtgccaacgtctacggctcctaccagtgttactgccggcgaggctaccagctcagcgatgtggatggagtcacctgtgaagacatcgacgagtgcgccctgcccaccgggggccacatctgctcctaccgctgcatcaacatccctggaagcttccagtgcagctgcccctcgtctggctacaggctggcccccaatggccgcaactgccaagacattgatgagtgtgtgactggcatccacaactgctccatcaacgagacctgcttcaacatccagggcggcttccgctgcctggccttcgagtgccctgagaactaccgccgctccgcagccacccgctgtgagcgcttgccttgccatgagaatcgggagtgctccaagctgcctctgagaataacctactaccacctctctttccccaccaacatccaagcgcccgcggtggttttccgcatgggcccctccagtgctgtccccggggacagcatgcagctggccatcaccggcggcaatgaggagggctttttcaccacccggaaggtgagcccccacagtggggtggtggccctcaccaagcctgtccccgagcccagggacttgctcctgaccgtcaagatggatctctctcgccacggcaccgtcagctcctttgtggccaagcttttcatctttgtgtctgcagagctctga

SEQ ID NO: 7 - protein, 703 aa, Homo sapiens
MERAAPSRRVPLPLLLLGGLALLAAGVDADVLLEACCADGHRMATHQKDCSLPYATESKECRMVQEQCCHSQLEELHCATGISLANEQDRCATPHGDNASLEATFVKRCCHCCLLGRAAQAQGQSCEYSLMVGYQCGQVFRACCVKSQETGDLDVGGLQETDKIIEVEEEQEDPYLNDRCRGGGPCKQQCRDTGDEVVCSCFVGYQLLSDGVSCEDVNECITGSHSCRLGESCINTVGSFRCQRDSSCGTGYELTEDNSCKDIDECESGIHNCLPDFICQNTLGSFRCRPKLQCKSGFIQDALGNCIDINECLSISAPCPIGHTCINTEGSYTCQKNVPNCGRGYHLNEEGTRCVDVDECAPPAEPCGKGHRCVNSPGSFRCECKTGYYFDGISRMCVDVNECQRYPGRLCGHKCENTLGSYLCSCSVGFRLSVDGRSCEDINECSSSPCSQECANVYGSYQCYCRRGYQLSDVDGVTCEDIDECALPTGGHICSYRCINIPGSFQCSCPSSGYRLAPNGRNCQDIDECVTGIHNCSINETCFNIQGGFRCLAFECPENYRRSAATLQQEKTDTVRCIKSCRPNDVTCVFDPVHTISHTVISLPTFREFTRPEEIIFLRAITPPHPASQANIIFDITEGNLRDSFDIIKRYMDGMTVGVVRQVRPIVGPFHAVLKLEMNYVVGGVVSHRNVVNVHIFVSEYWF

SEQ ID NO: 8 - DNA, 2112 bp, Homo sapiens
atggagcgcgccgcgccgtcgcgccgggtcccgcttccgctgctgctgctcggcggccttgcgctgctggcggccggagtggacgcggatgtcctcctggaggcctgctgtgcggacggacaccggatggccactcatcagaaggactgctcgctgccatatgctacggaatccaaagaatgcaggatggtgcaggagcagtgctgccacagccagctggaggagctgcactgtgccacgggcatcagcctggccaacgagcaggaccgctgtgccacgccccacggtgacaacgccagcctggaggccacatttgtgaagaggtgctgccattgctgtctgctggggagggcggcccaggcccagggccagagctgcgagtacagcctcatggttggctaccagtgtggacaggtcttccgggcatgctgtgtcaagagccaggagaccggagatttggatgtcgggggcctccaagaaacggataagatcattgaggttgaggaggaacaagaggacccatatctgaatgaccgctgccgaggaggcgggccctgcaagcagcagtgccgagacacgggtgacgaggtggtctgctcctgcttcgtgggctaccagctgctgtctgatggtgtctcctgtgaagatgtcaatgaatgcatcacgggcagccacagctgccggcttggagaatcctgcatcaacacagtgggctctttccgctgccagcgggacagcagctgcgggactggctatgagctcacagaggacaatagctgcaaagatattgacgagtgtgagagtggtattcataactgcctccccgattttatctgtcagaatactctgggatccttccgctgccgacccaagctacagtgcaagagtggctttatacaagatgctctaggcaactgtattgatatcaatgagtgtttgagtatcagtgccccgtgccctatcgggcatacatgcatcaacacagagggctcctacacgtgccagaagaacgtgcccaactgtggccgtggctaccatctcaacgaggagggaacgcgctgtgttgatgtggacgagtgcgcgccacctgctgagccctgtgggaagggacatcgctgcgtgaactctcccggcagtttccgctgcgaatgcaagacgggttactattttgacggcatcagcaggatgtgtgtcgatgtcaacgagtgccagcgctaccccgggcgcctgtgtggccacaagtgcgagaacacgctgggctcctacctctgcagctgttccgtgggcttccggctctctgtggatggcaggtcatgtgaagacatcaatgagtgcagcagcagcccctgtagccaggagtgtgccaacgtctacggctcctaccagtgttactgccggcgaggctaccagctcagcgatgtggatggagtcacctgtgaagacatcgacgagtgcgccctgcccaccgggggccacatctgctcctaccgctgcatcaacatccctggaagcttccagtgcagctgcccctcgtctggctacaggctggcccccaatggccgcaactgccaagacattgatgagtgtgtgactggcatccacaactgctccatcaacgagacctgcttcaacatccagggcggcttccgctgcctggccttcgagtgccctgagaactaccgccgctccgcagccacgctccagcaggagaagacagacacggtccgctgcatcaagtcctgccgccccaacgatgtcacatgcgtgttcgaccccgtgcacaccatctcccacaccgtcatctcgctgcctaccttccgcgagttcacccgccctgaagagatcatcttcctccgggccatcacgccaccgcatcctgccagccaggctaacatcatcttcgacatcacggaagggaacctgcgggactcttttgacatcatcaagcgttacatggacggcatgaccgtgggtgtcgtgcgccaggtgcggcccatcgtgggcccatttcatgccgtcctgaagctggagatgaactatgtggtcgggggcgtggtctcccaccgaaatgttgtcaacgtccacatcttcgtctctgagtactggttctga

SEQ ID NO: 9 - RNA, 24 nt, Artificial
  Synthetic oligonucleotide sequence
gcaagcgcucacagcggcugcaag

SEQ ID NO: 10 - RNA, 23 nt, Artificial
  Synthetic oligonucleotide sequence
cuugcuaagacuuuauuaacgcc

SEQ ID NO: 11 - RNA, 25 nt, Artificial
  Synthetic oligonucleotide sequence
auuuugucugaaacccuguaaagag

SEQ ID NO: 12 - RNA, 24 nt, Artificial
  Synthetic oligonucleotide sequence
cacuuggcgcacgacaccugggga

SEQ ID NO: 13 - RNA, 25 nt, Artificial
  Synthetic oligonucleotide sequence
ugaacuugauccacucacccucagg

SEQ ID NO: 14 - RNA, 24 nt, Artificial
  Synthetic oligonucleotide sequence
uuuucucuuggcggaagcugcaga

(19) United States
(12) Patent Application Publication
(10) Pub. No.: US 2011/0028408 A1
(43) Pub. Date: Feb. 3, 2011

(54) DIAGNOSTIC MOLECULE AND THERAPEUTIC TARGET

(75) Inventors: Janette Kay Burgess, New South Wales (AU); Justine Yeeman Lau, New South Wales (AU); Brian Gregory George Oliver, New South Wales (AU); Judith Lee Black, New South Wales (AU)

Correspondence Address: LOWE HAUPTMAN HAM & BERNER, LLP, 1700 DIAGONAL ROAD, SUITE 300, ALEXANDRIA, VA, 22314, US

(73) Assignee: CRC FOR ASTHMA AND AIRWAYS LTD, New South Wales (AU)

(21) Appl. No.: 12/740,805

(22) PCT Filed: Oct. 31, 2008
(86) PCT No.: PCT/AU2008/001618

§ 371 Date: Aug. 30, 2010

Related U.S. Application Data
(60) Provisional application No. 61/001,212, filed on Oct. 31, 2007.

(51) Int. Cl. A61K 31/7088; A61K 38/17; G01N 33/68; A61P 11/06; A61P 11/00

(52) U.S. Cl. 514/20.9; 514/44 A; 436/86; 435/7.1

(57) ABSTRACT

Provided herein are methods and compositions for regulating airway tissue remodelling and for treating or preventing conditions associated with airway inflammation and/or airway tissue remodelling. The methods comprise normalising fibulin-1 levels in said tissue relative to normal endogenous levels. In an embodiment the methods comprise administering to a subject in need thereof an effective amount of at least one agent capable of inhibiting the expression and/or activity of fibulin-1.

### FIELD OF THE INVENTION

The present invention relates generally to methods for modulating airway tissue remodelling, and in particular for treating or preventing conditions associated with aberrant airway tissue remodelling, such as asthma. More particularly, the invention relates to modulation of the levels and/or activity of fibulin-1 in airway tissue. The invention also relates to compositions useful for such methods. The present invention further relates to methods for diagnosing the occurrence or predisposition to the occurrence of unwanted airway tissue remodelling and conditions associated therewith, such as asthma. The present invention also relates to methods and compositions for the inhibition of wound repair, particularly in subjects suffering from conditions associated with aberrant airway tissue remodelling in which aberrant, excessive or otherwise unwanted tissue wound repair occurs.

### BACKGROUND OF THE INVENTION

Asthma is an episodic disease of the airways and is the most widespread chronic health problem in the Western world and is increasing in prevalence around the world. In Australia alone, it affects over 2 million individuals, including an estimated 1 in 4 children. Worldwide bronchial asthma is the most common chronic disease in childhood with an overall prevalence between 5 and 20 percent across all ages. The pathogenesis of asthma is suggested to be complex and is centred on an aberrant immune response to inhalant allergens that results in an inflammation of the airway wall together with an episodic constriction of the airways resulting in symptoms such as shortness of breath, wheezing, coughing, and life-threatening dyspnoea.

Asthma is also typically characterised by variable airflow obstruction that may be reversible (either spontaneously or with treatment), presence of airway hyperresponsiveness and chronic airway inflammation in which many cells and cellular elements play a role, in particular, mast cells, eosinophils, T lymphocytes, macrophages, neutrophils, and epithelial cells. All of these features need not be present in any given asthmatic patient. Although the absolute “minimum criteria” to establish a diagnosis of asthma is not widely agreed upon, the presence of airway hyperresponsiveness is a common finding in patients with current symptoms and active asthma.

In sensitive individuals, asthma symptoms can be triggered by inhaled allergens (allergy triggers), such as pet dander, dust mites, cockroach allergens, molds, or pollens. Asthma symptoms can also be triggered by respiratory infections, exercise, cold air, tobacco smoke and other pollutants, stress, food, or drug allergies. Aspirin and other non-steroidal anti-inflammatory medications provoke asthma in some patients. When an asthma attack occurs, the muscles of the bronchial tree become tight and the lining of the air passages swells, reducing airflow and producing the characteristic wheezing sound. Mucus production is increased. Most people with asthma exhibit periodic wheezing attacks separated by symptom-free periods. Some asthmatics exhibit chronic shortness of breath with episodes of increased shortness of breath.

Asthma is an inflammatory disease and is not simply due to excessive smooth muscle contraction. Asthma is a chronic disease of the airways, however it is unknown whether inflammation initiates asthma or whether asthma initiates inflammation. In the lungs of healthy individuals inflammation is a common occurrence, and is in fact necessary to maintain normal lung homeostasis through the removal of pathogens such as bacteria and viruses and pollutants which are present in the air. In the lungs of an asthmatic, an exaggerated response to irritants occurs which results in an increased tendency to produce excessive airway narrowing (hyperresponsiveness). Increased airway inflammation follows exposure to inducers such as allergens, viruses, exercise, or non-specific irritant inhalation. Increased inflammation leads to exacerbations characterised by dyspnoea, wheezing, cough, and chest tightness. Abnormal histopathologic lesions including oedema, epithelial cell desquamation, and inflammatory cell infiltration are found not only in severe asthma cases but even in patients with very mild asthma. One aspect of asthma etiology which remains relatively poorly understood is the occurrence of airway tissue remodelling. Pathologically, airway remodelling appears to have a variety of features that include an increase in smooth muscle mass, mucus gland hyperplasia, persistence of chronic inflammatory cellular infiltrates, alterations in extracellular matrix deposition and release of fibrogenic growth factors.

Asthma is therefore a disease in which inflammation of the airways causes airflow obstruction and airway hyperresponsiveness, and in which structural changes or ‘remodelling’ of the surface of the airways takes place. It is unclear if airway tissue remodelling is secondary to, and caused by, airway inflammation or if it is a separate phenomenon. Airway remodelling in diseases such as asthma is associated with hypertrophy and hyperplasia of cells such as airway smooth muscle cells and this can lead to a worsening of clinical symptoms. Further, tissue remodelling is a fundamental component of wound repair, which repair may become excessive in pathological conditions such as asthma also exacerbating symptoms.

Presently asthma treatment is typically aimed at avoiding known allergens and respiratory irritants and controlling symptoms and airway inflammation through medication. In terms of medication, this may include the use of short and/or long term treatment regimens. For short term ‘quick’ relief, for example during an asthma attack, short-acting bronchodilators (e.g. salbutamol, fenoterol, terbutaline, and albuterol) may be employed. For longer term control measures, medications include, for example, inhaled steroids, typically corticosteroids (e.g. triamcinolone acetonide, beclomethasone, flunisolide, fluticasone propionate, and budesonide) to prevent inflammation, leukotriene inhibitors (e.g. montelukast sodium, zafirlukast), long-acting bronchodilators (e.g. formoterol, salmeterol) to help keep airways open, a combination of corticosteroid and bronchodilator, using either separate inhalers or a single inhaler (e.g. fluticasone/salmeterol and budesonide/formoterol), and antibodies to neutralise immunoglobulin E (IgE) or interleukin-5 (IL-5) (e.g. omalizumab). Alternative current therapies aim to inhibit the constriction of airways by stimulating beta-2 receptors in the airways via short or long-acting beta-2 receptor agonists (“relievers” and “controllers” respectively).

However, existing therapies do not address the complex nature of the pathways that are activated in the airways during asthma on a molecular level. Rather they aim to either suppress only one or a few out of numerous disease mechanisms that promote aberrant immune responses or alleviate symptoms. Furthermore current therapies are commonly associated with significant side effects (for example in the case of steroid use) or tachyphylaxis (for example following administration of long-acting beta-2 receptor antagonists). Furthermore, despite significant progress in terms of understanding the cellular basis of asthma and the development of a range of treatment options, the cause of asthma is not known, nor has there been a cure developed.

Accordingly, there is not only a clear need for the development of effective therapies for the treatment of inflammatory conditions such as asthma, but also an ongoing need to pursue asthma-related research at the level of both understanding its cause and developing new treatment regimens which can contribute to expanding the existing range of therapeutic and prophylactic treatments which are available to the public.

### SUMMARY OF THE INVENTION

The present invention is predicated on the inventors' surprising finding that the extracellular matrix protein fibulin-1 is differentially expressed in airway smooth muscle cells from asthmatic and nonasthmatic individuals. Expression of fibulin-1 can be stimulated by pro-proliferative and pro-fibrotic factors such as foetal bovine serum and transforming growth factor β and fibulin-1 levels are increased in serum and bronchoalveolar lavage fluid from asthmatics. Further, the inventors demonstrate herein that TGFβ induced airways hyperresponsiveness in a murine model of asthma can be reduced by inhibiting the expression of fibulin-1. Moreover, as exemplified herein, the inventors have surprisingly identified that fibulin-1 promotes wound healing in airway smooth muscle cells in asthmatic patients and that silencing the expression of fibulin-1 abrogates this ability.

These findings have enabled the development of a method of reducing the incidence and/or severity of tissue remodelling in patients exhibiting airway diseases such as asthma. In the context of asthma these findings are extremely valuable in that they provide an adjunctive treatment regimen directed to minimising or reducing the occurrence of “scarring” of airway tissue. Accordingly, there is provided a means of reducing the severity of one of the more serious consequences of both managed and unmanaged forms of airway diseases such as asthma. The present inventors' findings also provide a novel means for diagnosing the occurrence of, or predisposition to, airway remodelling diseases such as asthma, based for example on an analysis of fibulin-1 mRNA or protein expression levels.

According to a first aspect of the present invention there is provided a method for treating or preventing a condition associated with airway inflammation and/or airway tissue remodelling, the method comprising administering to a subject in need thereof an effective amount of at least one agent capable of inhibiting the expression and/or activity of fibulin-1.

In an embodiment, the agent may be an antagonist of activity of the fibulin-1 polypeptide. The antagonist may be an antibody, such as a monoclonal antibody. In another embodiment, the agent may be a molecule(s) capable of inhibiting or suppressing expression of fibulin-1. Inhibition of expression may be at the level of the fibulin-1 nucleotide sequence and the inhibitor may be an antisense construct such as a small interfering RNA (siRNA), catalytic antisense construct, morpholino or other antisense oligonucleotide.

In an embodiment the fibulin-1 is isoform fibulin-1 B or fibulin-1 C.

The inhibition of the expression and/or activity of fibulin-1 may occur in one or more cells, typically abnormal cells implicated in the condition, and/or in the extracellular matrix associated with such cells.

The condition may be selected from, for example, asthma, chronic obstructive pulmonary disease (COPD), pulmonary fibrosis, cystic fibrosis and lymphangioleiomyomatosis (LAM). The subject may suffer from, or be predisposed to, the condition.

According to a second aspect the present invention provides a method for the treatment or prevention of asthma, the method comprising administering to a subject suffering from, or predisposed to, asthma an effective amount of at least one agent capable of inhibiting the expression and/or activity of fibulin-1.

According to a third aspect the present invention provides a method of regulating the occurrence of airway tissue remodelling in a subject, said method comprising modulating the level of fibulin-1 in airway tissue of the subject, wherein normalising fibulin-1 levels in said tissue relative to normal endogenous levels reduces the occurrence of, or inhibits, tissue remodelling.

Where normalising fibulin-1 levels relative to normal endogenous levels involves decreasing fibulin-1, levels of fibulin-1 may be normalised by administering an effective amount of one or more agents capable of decreasing the expression or production of fibulin-1.

In an embodiment the fibulin-1 is isoform fibulin-1 B or fibulin-1 C.

Where normalising fibulin-1 levels relative to normal endogenous levels involves increasing fibulin-1, levels of fibulin-1 may be increased by administering an effective amount of fibulin-1, a derivative, variant or homologue thereof, or an agent capable of increasing the expression or production of fibulin-1.

According to a fourth aspect the present invention provides a method for diagnosing a condition associated with airway inflammation and/or airway tissue remodelling, or susceptibility or predisposition thereto, in a subject, the method comprising determining the level of fibulin-1 in a fluid or airway tissue or cells of the subject.

The fluid may be serum or bronchoalveolar lavage fluid. The cells may be airway smooth muscle cells.

The method may comprise isolating fluid or airway cells from the subject and determining the level of expression of at least one fibulin-1 isoform in the fluid or airway cells, wherein the level of expression of the at least one fibulin-1 isoform is indicative of the condition, or a susceptibility or predisposition thereto.

Wherein airway cells are isolated, the cells may be stimulated in vitro prior to determination of fibulin-1 expression using one or more pro-proliferative or pro-fibrotic factors. By way of example, the pro-proliferative factor may be foetal bovine serum and the pro-fibrotic factor may be transforming growth factor-β.

The at least one fibulin-1 isoform may be fibulin-1 B or fibulin-1 C.

According to a fifth aspect the present invention provides the use of an agent capable of inhibiting the expression and/or activity of fibulin-1 in the airway tissue of a subject in the manufacture of a medicament for the treatment or prevention of a condition associated with airway inflammation and/or airway tissue remodelling.

According to a sixth aspect the present invention provides the use of an agent capable of inhibiting the expression and/or activity of fibulin-1 in the airway tissue of a subject for treating or preventing a condition associated with airway inflammation and/or airway tissue remodelling.

According to a seventh aspect the present invention provides the use of an agent capable of inhibiting the expression and/or activity of fibulin-1 in the airway tissue of a subject in the manufacture of a medicament for the treatment or prevention of asthma.

According to an eighth aspect the present invention provides the use of an agent capable of inhibiting the expression and/or activity of fibulin-1 in the airway tissue of a subject for treating or preventing asthma.

According to a ninth aspect the present invention provides a pharmaceutical composition for treating or preventing a condition associated with airway inflammation and/or airway tissue remodelling, the composition comprising at least one agent capable of inhibiting the expression and/or activity of fibulin-1 in airway tissue, optionally together with one or more pharmaceutically acceptable adjuvants, carriers and/or diluents.

According to a tenth aspect the present invention provides a pharmaceutical composition for treating or preventing asthma, the composition comprising at least one agent capable of inhibiting the expression and/or activity of fibulin-1 in airway tissue, optionally together with one or more pharmaceutically acceptable adjuvants, carriers and/or diluents.

According to an eleventh aspect the present invention provides a method for the treatment or prevention of a condition associated with airway inflammation and/or airway tissue remodelling in a subject, the method comprising modulating the level of fibulin-1 in the airway tissue of the subject wherein normalising fibulin-1 levels in said tissue relative to normal endogenous levels downregulates the occurrence of tissue remodelling.

According to a twelfth aspect the present invention provides a method for the treatment or prevention of asthma in a subject, said method comprising normalising fibulin-1 levels in the bronchial and/or bronchiolar tissue of the subject relative to normal endogenous levels wherein normalising said fibulin-1 level downregulates tissue remodelling in said bronchial and/or bronchiolar tissue.

According to a thirteenth aspect the present invention provides a method for inhibiting aberrant, excessive or otherwise unwanted wound repair in a tissue of a subject, wherein the subject suffers from or is predisposed to a condition associated with airway inflammation and/or airway tissue remodelling, the method comprising administering to the subject an effective amount of at least one agent capable of inhibiting the expression and/or activity of fibulin-1.

The condition associated with airway inflammation and/or airway tissue remodelling may be selected from, for example, asthma, chronic obstructive pulmonary disease (COPD), pulmonary fibrosis, cystic fibrosis and lymphangioleiomyomatosis (LAM).

The inhibition of wound repair may comprise inhibiting the expression and/or activity of fibulin-1 in one or more abnormal cells. The cells may be of any cell type, for example airway smooth muscle cells, epithelial cells or fibroblasts. Alternatively or in addition the inhibition of wound repair may comprise inhibiting the expression and/or activity of fibulin-1 in the extracellular matrix.

In an embodiment the agent is a molecule capable of inhibiting or suppressing expression of fibulin-1, for example an antisense oligonucleotide.

### BRIEF DESCRIPTION OF THE DRAWINGS

Embodiments of the present invention will now be described, by way of non-limiting example only, with reference to the accompanying drawings.

FIG. 1. The expression of mRNA encoding fibulin-1 normalised to the expression of the housekeeping gene 18S rRNA was measured using real time quantitative PCR. Samples were compared to 0.1% BSA values. 5% FBS and 10 ng/mL TGFβ significantly upregulated fibulin-1 mRNA expression (p<0.001 in both cases) in ASM cells isolated from asthmatic volunteers (n=up to 9). This increase was not observed in non-asthmatic volunteers (n=12).

FIG. 2. The expression of mRNA encoding fibulin-1A (A), fibulin-1 B (B), fibulin-1 C(C) and fibulin-1D (D) normalised to the expression of the housekeeping gene 18S rRNA was measured using real time quantitative PCR. Samples were compared to 0.1% BSA values. A. 5% FBS and 10 ng/mL TGFβ significantly downregulated fibulin-1A mRNA expression in ASM cells isolated from volunteers with asthma (p<0.001 in both cases, n=8) and non-asthma (p<0.001 in both cases, n=12). B. 10 ng/mL TGFβ significantly upregulated fibulin-1B mRNA expression in ASM cells isolated from asthmatic volunteers (p<0.001, n=8). C. 5% FBS and 10 ng/mL TGFβ significantly upregulated fibulin-1C in ASM cells isolated from asthmatic volunteers (p<0.001 for both cases, n=8). In contrast, 5% FBS significantly downregulated fibulin-1C in ASM cells isolated from non-asthmatic volunteers (p<0.001 for both cases, n=12). D. 5% FBS significantly downregulated fibulin-1D in ASM cells isolated from volunteers with asthma (p<0.001, n=8) and non-asthma (p<0.001, n=12). TGFβ significantly downregulated fibulin-1D only in ASM cells isolated from volunteers with asthma (p<0.001, n=8).

FIG. 3. A. Densitometric evaluation of the change in fibulin-1 in ASM cells from 4 non-asthmatic volunteers (left panel, unfilled bars) and 4 asthmatic volunteers (right panel; filled bars) using western blotting. 10 ng/mL TGFβ upregulated fibulin-1 protein in ASM cells only from asthmatic volunteers (p<0.05). Data expressed as fold change to non-stimulated cells. B. Densitometric evaluation of the amount of fibulin-1 contained in ASM cells stimulated with either 5% FBS or 10 ng/mL TGFβ from 4 asthmatic and 4 non-asthmatic volunteers using western blotting. Cell lysate from SKBR3 was used as a positive control. The expression of fibulin-1 protein levels were normalised to the SKBR3 and to the housekeeping protein GAPDH. Basally, there is no difference in fibulin-1 protein expression between ASM cells derived from asthmatic and non-asthmatic volunteers.

FIG. 4. A. Staining for fibulin-1 in ASM cells derived from one asthmatic volunteer compared to staining observed using an isotype control in the presence of either 0.1% (w/v) BSA, 5% (v/v) FBS, or 10 ng/mL TGFβ. The intensity of fibulin-1 in ASM cells stimulated with 10 ng/mL TGFβ is increased. These immunohistochemical images represent all four asthmatic volunteers tested. B. Staining for fibulin-1 in ASM cells derived from one non-asthmatic volunteer compared to staining observed using an isotype control in the presence of either 0.1% (w/v) BSA, 5% (v/v) FBS, or 10 ng/mL TGFβ. These immunohistochemical images represent all five non-asthmatic volunteers tested.

FIG. 5. Staining for fibulin-1 in bronchial biopsies treated with TGFβ by immunohistochemistry. Biopsies were derived from (A) one non-asthmatic and (B) one asthmatic participant and treated using 0.1% BSA or 10 ng/mL TGFβ for 24 hours. Morphology of bronchial biopsies shown by haematoxylin and eosin staining. The staining for fibulin-1 was compared with the level of staining observed in the presence of an isotype control. Images were taken at 40× and are representative of the three non-asthmatic and three asthmatic derived biopsies tested. A. TGFβ did not alter the staining intensity for fibulin-1 in non-asthmatic derived biopsies. B. The intensity of fibulin-1 in the asthmatic derived biopsies stimulated with 10 ng/mL TGFβ is increased.

FIG. 6. A. Co-localisation of fibulin-1 and fibronectin in ASM cells basally. Cells were derived from a non-asthmatic and an asthmatic participant and treated with 0.1% BSA for 24 hours. Superimposed image represents the addition of fibulin-1 and fibronectin. Images were taken at 40× and are representative of the five non-asthmatic and four asthmatic derived cell lines studied B. Co-localisation of fibulin-1 and fibronectin in bronchial ring tissue sections treated with TGFβ analysed by immunohistochemistry. Rings were derived from a non-asthmatic participant and treated with 10 ng/mL TGFβ for 24 hours. Morphology of bronchial rings shown by haematoxylin and eosin staining. The staining for fibulin-1 and fibronectin were compared with the level of staining observed in the presence of an isotype. Images were taken at 20× and are representative of the seven non-asthmatic derived samples tested. Fibulin-1 and fibronectin co-localise within the basement membrane and ASM bulk.

FIG. 7. A. Fibulin-1 deposition in the ECM produced by ASM cells. 10 ng/mL TGFβ did not change fibulin-1 deposition in the ECM from non-asthmatic (n=10) volunteers (left panel; unfilled bars), but increased deposition in ECM from asthmatic (n=10) volunteers (p<0.05) (right panel; filled bars) measured using an ECM ELISA. Data expressed as fold change compared to non stimulated cells. B. Fibronectin deposition in the ECM produced by ASM cells. 10 ng/mL TGFβ did not change fibronectin deposition in the ECM from non-asthmatic (n=10) volunteers (left panel; unfilled bars), but increased deposition in ECM from asthmatic (n=10) volunteers (p<0.01) (right panel; filled bars) measured using an ECM ELISA. Data expressed as fold change compared to non stimulated cells.

FIG. 8. Densitometric evaluation of the amount of fibulin-1 contained in the supernatant of ASM cells treated with either 0.1% BSA, 5% FBS or 10 ng/mL TGFβ from 5 asthmatics and 4 non-asthmatics using dot blot. Data expressed in relation to the positive control.

FIG. 9. A. Densitometric evaluation of the amount of fibulin-1 contained in serum of 21 asthmatic volunteers receiving no treatment, 15 asthmatic volunteers receiving corticosteroids and 20 non-asthmatics using dot blotting techniques. Fibulin-1 in serum of asthmatic volunteers is higher than that found in non-asthmatic volunteers (p<0.01) (left panel). However, corticosteroids did not alter fibulin-1 in serum taken from asthmatic volunteers (right panel). Data expressed in relation to a positive control. B. Densitometric evaluation of the amount of fibulin-1 contained in the BALF of 20 asthmatic and 11 non-asthmatic volunteers using dot blotting techniques. Fibulin-1 in BALF of asthmatics receiving no corticosteroid treatment is three times that found in non-asthmatic volunteers (p<0.01) (left panel). However, corticosteroids did not alter fibulin-1 in BALF taken from asthmatic volunteers (right panel). Data expressed in relation to a positive control.

FIG. 10. A. TFGβ increased specific airway resistance to methacholine in comparison to naïve (saline) treated animals. Concurrent treatment with an anti-fibulin-1 antisense oligonucleotide mixture (AO) and TGFβ inhibited the development of specific airway resistance to methacholine (20 mg/ml), i.e. there was no difference in the specific airway resistance to methacholine in comparison to naive animals. The mice which had received concurrent treatment with a scrambled AO and TGFβ had increased specific airway resistance to methacholine (20 mg/ml) in comparison to naïve animals. In the absence of TGFβ, mice treated with AO or scrambled AO had no alteration in their specific airway resistance to methacholine. *** p<0.01 in comparison to saline/saline. B. TGFβ alone decreased specific airway compliance to methacholine in comparison to naïve (saline) treated animals. The mice which had received concurrent treatment with AO and TGFβ had no alteration in specific airway compliance to methacholine (20 mg/ml) in comparison to naïve animals. The mice which had received concurrent treatment with a scrambled AO and TGFβ had decreased specific airway compliance to methacholine (20 mg/ml) in comparison to naïve animals. In the absence of TGFβ, mice treated with AO or scrambled AO had no alteration in their decreased specific airway compliance to methacholine. *** p<0.01 in comparison to saline/saline.

FIG. 11. A. Wound healing rates of ASM cells derived from non-asthmatics and asthmatics using a wound healing assay. ASM cells were derived from non-asthmatic (n=4) and asthmatic (n=3) volunteers and treated with 0.1% BSA over 72 hours. Data are expressed as a percentage of original wound area closed and shown as mean±SEM and compared with 0 hours. Wound healing is increased in asthmatic derived ASM cells 24 hours post wound induction (P<0.05) B & C. Wound healing rates of ASM cells treated with TGFβ using a wound healing assay. ASM cells were derived from (B) asthmatic volunteers (n=3) and (C) non-asthmatic volunteers (n=4) and treated with 0.1% BSA or 10 ng/mL TGFβ over 72 hours. Data are expressed as a percentage of original wound area closed and shown as mean±SEM and compared with 0 hours. 10 ng/mL TGFβ increased wound healing rates of both asthmatic and non-asthmatic derived ASM cells 48 hours post wound induction (P<0.05, P<0.01 respectively).

FIG. 12. The effect of fibulin-1 on rates of wound healing of ASM cells treated with TGFβ. Cells were derived from non-asthmatic (n=4) and asthmatic (n=3) volunteers. ASM cells were transfected with 200 nM scrambled antisense or JSR1307 for 72 hours and treated with 10 ng/mL TGFβ over 72 hours. Data are expressed as a percentage of original wound area closed and shown as mean±SEM. Statistical values compared at 72 hours to time matched values of non-asthmatic, TGFβ, scrambled antisense. 10 ng/mL TGFβ stimulation increased the rate of wound healing only in asthmatic derived ASM cells. By silencing fibulin-1 in the asthmatic derived ASM cells, the increased rate was abolished and returned to that of non-asthmatic derived ASM cells.

FIG. 13. The effect of fibulin-1 in asthmatic derived ECM on wound healing rate of ASM cells. ASM cells were derived from non-asthmatic (n=8) and asthmatic (n=4) participants and seeded on an ECM produced by asthmatic participants (n=4). ECM were transfected using either 200 nM scrambled antisense or JSR1307 for 72 hours and cells were treated using either (A) 0.1% BSA or (B) 10 ng/mL TGFβ over 72 hours. Data expressed as a percentage of area closed compared with its initial wound size and shown as mean±SEM. Statistical values compared at 72 hours to time matched values of non-asthmatic cells seeded on asthmatic ECM transfected with scrambled antisense. A & B Both basally and in the presence of 10 ng/mL TGFβ, faster wound closure was observed in the asthmatic cells seeded on asthmatic ECM (P<0.001 in both cases). A. By silencing fibulin-1 in the asthmatic ECM, the increased rate was abolished such that the rate of wound closure of asthmatic ASM cells returned to that of non-asthmatic ASM cells. B. By silencing fibulin-1 in the asthmatic ECM, the rate of wound healing of both asthmatic and non-asthmatic derived ASM cells was further decreased when compared with non-asthmatic cells seeded on an asthmatic ECM (P<0.05 in both cases).

FIG. 14. The effect of fibulin-1 in ASM and ECM on wound healing rates. ASM cells were derived from (A & B) non-asthmatic (n=4) and (C & D) asthmatic (n=2) volunteers and seeded on an ECM produced by asthmatic (n=4) volunteers. Both ECM and ASM cells were treated with no antisense or transfected with 200 nM JSR1307 for 72 hours. ASM cells were further treated with (A & C) 0.1% BSA or (B & D) 10 ng/mL TGFβ over 72 hours. Data are expressed as a percentage of area closed compared with its initial wound area and shown as mean±SEM. Wound healing rates are decreased when ASM cells and ECM are both silenced for fibulin-1 (P<0.05 in all cases).

The present specification contains amino acid and nucleotide sequence information prepared using the programme PatentIn Version 3.1, presented herein after the bibliography. Each amino acid and nucleotide sequence is identified in the sequence listing by the numeric indicator <210> followed by the sequence identifier (eg. <210>1, <210>2, etc). The length, type of sequence (amino acid, DNA, etc.) and source organism for each sequence is indicated by information provided in the numeric indicator fields <211>, <212> and <213>, respectively. Amino acid and nucleotide sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing.

Specifically, the amino acid sequences set forth in SEQ ID NOs: 1, 3, 5 and 7 represent the sequences of human fibulin-1, isoforms A (GenBank Accession No. NM_{—}006487), B (GenBank Accession No. NM_{—}006485), C (GenBank Accession No. NM_{—}001996) and D (GenBank Accession No. NM_{—}006486) respectively. The corresponding nucleotide sequences for these isoforms are represented in SEQ ID Nos: 2, 4, 6 and 8. The nucleotide sequences provided in SEQ ID NOs: 9 to 14 represent antisense oligonucleotide sequences as described herein.

### DETAILED DESCRIPTION OF THE INVENTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

In the context of this specification, the term “activity” as it pertains to fibulin-1 means any cellular or extracellular function, action, effect or influence exerted by fibulin-1, either by a nucleic acid sequence or fragment thereof encoding fibulin-1, or by the fibulin-1 gene product itself or any fragment thereof. “Activity” may therefore relate to the activity of fibulin-1 on a gene or gene product acting downstream thereof and the term “activity” is therefore interpreted as also encompassing the fibulin-1 expression and activities of these downstream genes and gene products.

As used herein, an “agent capable of inhibiting the expression and/or activity” of fibulin-1 or an “inhibitor” of fibulin-1 refers to any agent or action capable of inhibiting either or both the expression or activity of fibulin-1, either directly or indirectly. Accordingly the inhibitor may operate directly or indirectly on fibulin-1 (any one or more isoforms thereof), the fibulin-1 mRNA (any one or more splice variants thereof) or the fibulin-1 gene, or alternatively act via the direct or indirect inhibition of any one or more components of a fibulin-1-associated pathway. Such components may be molecules activated, inhibited or otherwise modulated prior to, in conjunction with, or as a consequence of fibulin-1 activity. Thus, the inhibitor may operate to prevent transcription, translation, post-transcriptional or post-translational processing or otherwise inhibit the activity of fibulin-1 or a component of a fibulin-1-associated pathway in any way, via either direct or indirect action. The inhibitor may for example be nucleic acid, peptide, any other suitable chemical compound or molecule or any combination of these. It will be understood that in indirectly impairing the activity of fibulin-1 or a component of a fibulin-1-associated pathway, the inhibitor may effect the activity of molecules which are themselves subject to regulation or modulation by fibulin-1 or a component of a fibulin-1-associated pathway.

As used herein the term “effective amount” includes within its meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The term “expression” as used herein refers interchangeably to expression of a gene or gene product, including the encoded protein. Expression of a gene may be determined, for example, by measuring the production of messenger RNA (mRNA) transcript levels. Expression of a polypeptide gene product may be determined, for example, by immunoassay using an antibody(ies) that bind with the polypeptide.

The term “inhibiting” and variations thereof such as “inhibition” and “inhibits” as used herein do not necessarily imply the complete inhibition of the specified event, activity or function. Rather, the inhibition may be to an extent, and/or for a time, sufficient to produce the desired effect. Inhibition may be prevention, retardation, reduction or otherwise hindrance of the event, activity or function. Such inhibition may be in magnitude and/or be temporal in nature. In particular contexts, the terms “inhibit” and “prevent”, and variations thereof may be used interchangeably.

As used herein the term “normalising fibulin-1 levels” refers to any means of modulating the amount, expression or activity of fibulin-1 in one or more cells.

As used herein the term “fibulin-1” refers to the secreted glycoprotein also known as fbln1, fbln, or otthump00000028589. In humans, four isoforms of fibulin-1 are known (A, B, C and D) and the term “fibulin-1” in the context of the present specification may be used in relation to any one or all of these isoforms or to nucleic acid molecules encoding the same. Thus the term “fibulin-1” as used herein encompasses all forms of the molecule as well as functional derivatives or homologues thereof, all isoforms, functional mutants and polymorphic variants thereof.

As used herein the term “polypeptide” means a polymer made up of amino acids linked together by peptide bonds. The terms “polypeptide” and “protein” are used interchangeably herein, although for the purposes of the present invention a “polypeptide” may constitute a portion of a full length protein.

The term “polynucleotide” as used herein refers to a single- or double-stranded polymer of deoxyribonucleotide, ribonucleotide bases or known analogues or natural nucleotides, or mixtures thereof. In some contexts in the present specification the terms “polynucleotide” and “nucleic acid molecule” are used interchangeably.

As used herein the term “subject” includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Typically, the mammal is human or a laboratory test animal. Even more typically, the mammal is a human.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery. Similarly, “prevention” dose not necessarily mean that the subject will not eventually contract a particular condition or disease. Rather, “prevention” encompasses reducing the severity of, or delaying the onset of, a particular condition or disease. In the context of some conditions, such as asthma, methods of the present invention involve “treating” the condition in terms of reducing or eliminating the occurrence of a highly undesirable and irreversible outcome of the progression of the condition but may not of itself prevent the initial occurrence of the condition, for example the occurrence of an asthma attack. Accordingly, treatment and prevention include amelioration of the symptoms of a particular condition or preventing or otherwise reducing the risk of developing a particular condition.

Fibulin-1 is a secreted glycoprotein that forms a component of, and assists in stabilising, the extracellular matrix (ECM). Fibulin-1 has been shown to associate in vivo with a variety of other proteins including fibronectin, laminin and fibrinogen as well as proteoglycans such as aggrecan. Fibulin-1 is thought to play a role in mediating cell adhesion, cell migration and cell differentiation. For example, it has been shown to mediate platelet adhesion by virtue of binding to fibrinogen. Fibulin-1 is a member of a family of secreted glycoproteins characterised by the presence of repeated epidermal growth factor-like domains. Four isoforms of fibulin-1 have been identified to date in humans, designated fibulin-1A, -1B, -1 C and -1D. These isoforms are splice variants which possess different C-terminal sequences.

Little is known about the effects of disregulation of fibulin-1 expression or activity, although recently fibulin-1 has been associated with several cancers including ovarian cancer and breast cancer.

As disclosed and exemplified herein, the present inventors have for the first time identified that fibulin-1 is differentially expressed in airway tissue between asthmatic and nonasthmatic individuals. The inventors have also found that fibulin-1 levels are increased in both serum and bronchoalveolar lavage fluid from asthmatics as compared to nonasthmatics, and that the expression of fibulin-1 can be significantly stimulated by pro-proliferative and pro-fibrotic factors such as foetal bovine serum (FBS) and transforming growth factor-β (TGFβ). Also disclosed herein is in vivo evidence that the inhibition of fibulin-1 in a murine model of asthma can reduce airways hyperresponsiveness. Moreover, as exemplified herein, the inventors have surprisingly identified that fibulin-1 promotes wound healing in airway smooth muscle cells in asthmatic patients and that silencing the expression of fibulin-1 abrogates this ability.

These findings therefore offer not only a novel diagnostic tool for the identification of conditions associated with aberrant airway tissue remodelling such as asthma and the identification of predisposition to such conditions, but also a new therapeutic approach to the treatment of such conditions. Tissue remodelling is the unwanted and undesirable outcome of some airway diseases such as asthma, chronic obstructive pulmonary disease (COPD), pulmonary fibrosis including idiopathic pulmonary fibrosis, cystic fibrosis and lymphangioleiomyomatosis (LAM). Accordingly, in the absence of the development of cures for these diseases, the development by the present inventors of means to reduce tissue remodelling is a crucial finding enabling the rational design of therapeutic and prophylactic methods for reducing the occurrence or severity of this highly undesirable and irreversible outcome of many airway diseases.

Accordingly, one aspect of the present invention provides a method for treating or preventing a condition associated with airway inflammation and/or airway tissue remodelling, the method comprising administering to a subject in need thereof an effective amount of at least one agent capable of inhibiting the expression and/or activity of fibulin-1.

Other aspects of the invention provide methods for the diagnosis of conditions associated with airway inflammation and/or airway tissue remodelling, or susceptibility or predisposition thereto, comprising determining the level of fibulin-1 in a fluid or airway tissue or cells of a subject. The fluid may be serum or bronchoalveolar lavage fluid. The cells may be airway smooth muscle cells.

Embodiments of the invention also provide pharmaceutical compositions for use in accordance with the methods of the invention.

As detailed herein, embodiments of the present invention are applicable to the treatment, prevention and/or diagnosis of conditions associated with aberrant airway inflammation and/or airway tissue remodelling. Such conditions include, but are not limited to, asthma, chronic obstructive pulmonary disease (COPD), pulmonary fibrosis, cystic fibrosis and lymphangioleiomyomatosis (LAM).

Without limiting the invention to any one theory or mode of action, it is hypothesised that fibulin-1 may play a role, directly or indirectly, in airway tissue remodelling processes, which processes underlie a number of conditions such as asthma. Accordingly, the invention contemplates methods of regulating the occurrence of airway tissue remodelling, the methods comprising modulating the level of fibulin-1 in airway tissue of a subject, wherein normalising fibulin-1 levels in the tissue relative to normal endogenous levels downregulates the occurrence of tissue remodelling.

Reference to regulating the “occurrence” of airway tissue remodelling by modulating fibulin-1 levels should be understood to mean that fibulin-1 may not of itself directly act on the cellular events which are generally understood to constitute “tissue remodelling”, although this is not excluded, but may act on a related cellular event which in turn impacts on the progression of one or more tissue remodelling pathologies. To this end, it should also be understood that reference to “tissue remodelling” is intended as a reference to any one or more of the cellular events and pathological outcomes which constitute the phenomenon of tissue remodelling. Whilst airway tissue remodelling is not fully understood, at the pathological level it is generally understood to encompass one or more of an increase in smooth muscle mass, mucus gland hyperplasia, persistence of chronic inflammatory cellular infiltrates, release of fibrogenic growth factors, collagen deposition, thickening of the lamina reticularis and increased extracellular matrix deposition. Reference to “tissue remodelling” should therefore be understood as reference to any one or more of these events since the range of pathologies which are associated with airway tissue remodelling in the context of one disease condition relative to another may differ.

By “airway tissue” is meant the tissue of the passages which run from the back of the mouth and nose into the lungs, together with the alveoli. The largest of these passages is the trachea (also known as the “windpipe”). In the chest, the trachea divides into two smaller passages termed the bronchi, each of these being further characterised by three regions termed the primary bronchus, secondary bronchus and tertiary bronchus. Each bronchus enters one lung and divides further into narrower passages termed the bronchioles. The terminal bronchiole supplies the alveoli. This network of passages are often colloquially termed the “bronchial tree” and, in the context of asthma, undergo inflammation, muscle constriction and swelling of their lining leading to a reduction in airflow into and out of the lungs. It is this tissue which also ultimately undergoes remodelling, thereby leading to still further complications in terms of the irreversible reduction of lung functioning.

Reference to “normal endogenous levels” should be understood as a reference to the level of fibulin-1 which is expressed in the airway tissue of a subject who is not suffering from nor is predisposed to a condition associated with aberrant airway inflammation and/or airway tissue remodelling. It would be appreciated by the person of skill in the art that this “normal level” is likely to correspond to a range of levels, as opposed to a singularly uniform discrete level, due to differences between cohorts of individuals. By “cohort” is meant a cohort characterised by one or more features which are also characteristic of the subject who is undergoing treatment. These features include, but are not limited to, age, gender or ethnicity, for example. Accordingly, reference herein to modulating fibulin-1 levels relative to normal endogenous levels is a reference to increasing or decreasing airway tissue fibulin-1 levels relative to either a discrete fibulin-1 level which may have been determined for normal individuals who are representative of the same cohort as the individual being treated or relative to a defined fibulin-1 level range which corresponds to that expressed by a population of individuals corresponding to those from a range of different cohorts.

In terms of modulating fibulin-1 levels, embodiments of the present invention provide for the downregulation of airway tissue fibulin-1 levels in order to approach the normal endogenous levels. To this end, however, it should be understood that the subject's fibulin-1 level need not necessarily be fully normalised in order to achieve the desired outcome, although complete normalisation is typically desirable. Merely partially decreasing fibulin-1 levels may at least ameliorate the incidence or severity of a condition in the subject. It should also be understood that the method of the present invention may be applied transiently or in an ongoing manner depending on the requirements of the particular situation. Further, it will be appreciated that there may be circumstances in which it is desirable or beneficial to reduce levels of fibulin-1 beyond normal endogenous levels. Such reduction of fibulin-1 levels is contemplated and encompassed by the present application.

As would be appreciated by those skilled in the art in some circumstances it may be desirable to induce or upregulate the occurrence of airway inflammation and/or tissue remodelling, for example in an in vitro model or an animal model, in order to facilitate an outcome such as providing a system for screening for the effectiveness of adjunctive therapies, prophylactic therapies or for otherwise facilitating the ongoing analysis of airway tissue remodelling. To this end, one may achieve this outcome by increasing the endogenous fibulin-1 levels of the subject airway tissue. Reference to “increasing” in this regard should be understood to have an analogous meaning to “normalising” in that said increase may be partial or total and will depend on the extent to which one is seeking to facilitate the occurrence of the desired event.

Also provided by the present invention are methods of inhibiting wound repair in a tissue of a subject suffering from, or predisposed to, a condition associated with airway inflammation and/or airway tissue remodelling, and wherein the wound healing is aberrant, excessive or otherwise unwanted. Typically, where the inhibition of wound repair involves the inhibition of fibulin-1 expression and/or activity, the method comprises administering to the subject an effective amount of at least one agent capable of inhibiting the expression and/or activity of fibulin-1.

Typically in accordance with embodiments of the invention wound repair is inhibited in one or more abnormal cells in the subject and/or in the extracellular matrix associated with such cells. In the present context, abnormal cells are cells, of any cell type, that are implicated in, or otherwise associated with the development, progression or symptoms of a disease condition of the airways as described herein. By way of example, the cells may be airway smooth muscle cells, epithelial cells or fibroblasts, although those skilled in the art will appreciate that the embodiments of the invention are not limited thereto. Where the expression or activity of fibulin-1 is to be inhibited, embodiments of the invention contemplate such inhibition in either or both of the cells and the extracellular matrix associated with the cells.

Those skilled in the art will appreciate that methods of the present invention may be performed in vivo, ex vivo or in vitro. Although methods are typically to therapeutically or prophylactically treat an individual in vivo, it should nevertheless be understood that it may be desirable that a method of the invention be applied in an ex vivo or in vitro environment, such as in the contexts detailed above.

Particular embodiments of the invention contemplate the administration of one or more agents capable of inhibiting or reducing the expression and/or activity of fibulin-1. Such inhibitors may directly or indirectly effect fibulin-1 expression and may act at the level of the fibulin-1 gene or any product thereof including fibulin-1 mRNA (precursor or mature message) or fibulin-1 polypeptide. The inhibitor may be a proteinaceous or non-proteinaceous molecule that modulates the transcription and/or translation of the fibulin-1 gene or a functional portion thereof (such as a promoter region), or alternatively that modulates the transcription and/or translation of an alternative gene or functional portion thereof, which alternative gene or gene product directly or indirectly modulates the expression of fibulin-1. The inhibitory agent may be an antagonist. Antagonists may be any compound capable of blocking, inhibiting or otherwise preventing fibulin-1 from carrying out its normal biological function. For the present purposes, the term “antagonist” is used hereinafter to refer to inhibitors of fibulin-1 activity and fibulin-1 expression.

A variety of suitable antagonists may be employed and the scope of the invention is not limited by the selection of any one particular molecule or compound. Suitable antagonists include antibodies, such as monoclonal antibodies, and antisense nucleic acids which prevent transcription or translation of fibulin-1 genes or mRNA. Modulation of expression may also be achieved utilising antigens, RNA, ribosomes, DNAzymes, aptamers, antibodies or molecules suitable for use in cosuppression.

Suitable antibodies include, but are not limited to polyclonal, monoclonal, chimeric, humanised, single chain, Fab fragments, and an Fab expression library. Antibodies may act as antagonists of fibulin-1 polypeptides, or fragments or analogues thereof. Preferably antibodies are prepared from discrete regions or fragments of the fibulin-1 polypeptide. Methods for the generation of suitable antibodies will be readily appreciated by those skilled in the art. For example, a suitable monoclonal antibody may be prepared using the hybridoma technology described in Antibodies-A Laboratory Manual, Harlow and Lane, eds., Cold Spring Harbor Laboratory, N.Y. (1988), the disclosure of which is incorporated herein by reference.

Monoclonal antibodies capable of inhibiting fibulin-1 activity are known. For example the mouse anti-human fibulin-1 monoclonal antibodies P5B1, P3B4, P1B2, MEM-2 and 3A11 have been described previously (see Argraves et al., 1990 and Pupa et al., 2003, the disclosures of which are incorporated herein by reference). The use of such antibodies in accordance with the present invention is contemplated.

Suitable antisense constructs for use in accordance with the present invention include antisense oligonucleotides, small interfering RNAs (siRNAs) and catalytic antisense nucleic acid constructs. Suitable antisense oligonucleotides may be prepared by methods well known to those of skill in the art. Typically oligonucleotides will be chemically synthesized on automated synthesizers. Those skilled in the art will readily appreciate that antisense oligonucleotides need not display 100% sequence complementarity to the target sequence. One or more base changes may be made such that less than 100% complementarity exists whilst the oligonucleotide retains specificity for its target and retains antagonistic activity against this target. Suitable antisense oligonucleotides include morpholinos where nucleotides comprise morpholine rings instead of deoxyribose or ribose rings and are linked via phosphorodiamidate groups rather than phosphates. By way of example only, two oligonucleotides capable of inhibiting fibulin-1 expression in airway smooth muscle cells are represented by the sequences of SEQ ID NOs:9 and 10.

An alternative antisense technology, known as RNA interference (RNAi), see, eg. Chuang et al. (2000) PNAS USA 97: 4985) may be used, according to known methods in the art (for example Hammond et al. (2000)Nature 404: 293-296; Bernstein et al. (2001)Nature 409: 363-366; Elbashir et al (2001)Nature 411: 494-498; WO 99/49029 and WO 01/70949, the disclosures of which are incorporated herein by reference), to inhibit the expression or activity of nucleic acid molecules encoding fibulin-1. RNAi refers to a means of selective post-transcriptional gene silencing by destruction of specific RNA by small interfering RNA molecules (siRNA). The siRNA is generated by cleavage of double stranded RNA, where one strand is identical to the message to be inactivated. Double-stranded RNA molecules may be synthesised in which one strand is identical to a specific region of the fibulin-1 transcript and introduced directly. Alternatively corresponding dsDNA can be employed, which, once presented intracellularly is converted into dsRNA. Methods for the synthesis of suitable molecules for use in RNAi and for achieving post-transcriptional gene silencing are known to those of skill in the art.

A further means of inhibiting the expression or activity of fibulin-1 may be involve introducing catalytic antisense nucleic acid constructs, such as ribozymes, which are capable of cleaving fibulin-1 mRNA transcripts. Ribozymes are targeted to and anneal with a particular sequence by virtue of two regions of sequence complementarity to the target flanking the ribozyme catalytic site. After binding the ribozyme cleaves the target in a site-specific manner. The design and testing of ribozymes which specifically recognise and cleave fibulin-1 mRNA sequences can be achieved by techniques well known to those in the art (for example Lieber and Strauss, (1995) Mol. Cell. Biol. 15:540-551, the disclosure of which is incorporated herein by reference).

If desired, agents for use in accordance with the present invention may be fused to other compounds, including peptides, polypeptides or other proteinaceous or non-proteinaceous molecules. For example, agents may be fused to molecules to facilitate localisation to the airway tissue.

Screening for suitable modulatory agents for use in accordance with the present invention may be achieved by any one of several suitable methods including, but in no way limited to, contacting a cell comprising the fibulin-1 gene or functional equivalent or derivative thereof with an agent and screening for the modulation of fibulin-1 protein production or functional activity, modulation of the expression of a nucleic acid molecule encoding fibulin-1 or modulation of the activity or expression of a downstream fibulin-1 cellular target. Detecting such modulation can be achieved utilising techniques such as Western blotting, electrophoretic mobility shift assays and/or the readout of reporters such as luciferases, chloramphenicol acetyltransferase (CAT) and the like.

Agents for use in accordance with the invention may modulate the expression or activity of any one or more fibulin-1 isoform. For example in humans, four isoforms have been identified to date, fibulin-1A, fibulin-1B, fibulin-1C and fibulin-1D. The amino acid sequences of these human isoforms are provided herein in SEQ ID Nos:1, 3, 5 and 7, respectively. The encoding nucleotide sequences are provided herein in SEQ ID Nos:2, 4, 6 and 8, respectively. Antagonists for use in accordance with the invention may therefore target, bind to or otherwise interact with any one or more of these sequences wherein the fibulin-1 to be modulated is derived from humans.

### Pharmaceutical Compositions

Agents may be administered in accordance with the present invention in the form of pharmaceutical compositions, which compositions may comprise one or more pharmaceutically acceptable carriers, excipients or diluents. Such compositions may be administered in any convenient or suitable route such as by parenteral, oral, nasal or topical routes. Typically, administration is via the respiratory route. In circumstances where it is required that appropriate concentrations of the desired agent are delivered directly to the site in the body to be treated, administration may be regional rather than systemic. Regional administration provides the capability of delivering very high local concentrations of the desired agent to the required site and thus is suitable for achieving the desired therapeutic or preventative effect whilst avoiding exposure of other organs of the body to the compound and thereby potentially reducing side effects.

It will be understood that the specific dose level of a composition of the invention for any particular individual will depend upon a variety of factors including, for example, the activity of the specific agents employed, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. Considering a patient, for example, from about 0.1 mg to about 1 mg of agent may be administered per kilogram of body weight per day. Dosage regimens may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Pharmaceutical forms suitable for injectable use include sterile aqueous solutions (where water soluble) or dispersions and sterile powders for the extemporaneous preparation of sterile injectable solutions or dispersions. The formulation must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filter sterilisation. Generally, dispersions are prepared by incorporating the various sterilised active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The present invention contemplates combination therapies, wherein agents as described herein are coadministered with other suitable agents which may facilitate the desired therapeutic or prophylactic outcome. For example, in the context of asthma, one may seek to maintain ongoing anti-inflammatory therapies in order to control the incidence of inflammation whilst employing agents in accordance with embodiments of the present invention. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

### Fibulin-1 Administration

Embodiments of the invention contemplate the administration of fibulin-1, or derivatives, variants or homologues thereof. The fibulin-1 may be derived from humans and may comprise an amino acid sequence as set forth in any one of SEQ ID Nos: 1, 3, 5 or 7, or be encoded by a polynucleotide comprising a nucleotide sequence as set forth in any one of SEQ ID NOS: 2, 4, 6 or 8. The fibulin-1 may be administered as a polypeptide or polynucleotide. Accordingly, also envisaged is the administration of a polynucleotide comprising a nucleotide sequence as set forth in any one of SEQ ID NOS: 2, 4, 6 or 8. The present invention also contemplates the use of derivatives, variants and homologues of human fibulin-1.

The fibulin-1 polynucleotide may be natural, recombinant or synthetic and may be obtained by purification from a suitable source or produced by standard recombinant DNA techniques such as those well known to persons skilled in the art, and described in, for example, Sambrook et al., Molecular Cloning: a Laboratory Manual, Cold Spring Harbor Laboratory Press (the disclosure of which is incorporated herein by reference). Where a polynucleotide encoding fibulin-1 is administered, the polynucleotide is typically present in a vector operably linked to suitable regulatory sequences capable of providing for the expression of the coding sequence by a cell. The term “regulatory sequence(s)” includes promoters and enhancers and other expression regulation signals. These may be selected to be compatible with the cell for which the expression vector is designed. Mammalian promoters, such as β-actin promoters and the myosin light chain promoter may be used. However, other promoters may be adopted to achieve the same effect. These alternate promoters are generally familiar to the skilled addressee.

Derivatives of fibulin-1 include functional fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, functionally active regions of the molecule which may be produced by synthetic or recombinant means well known to those skilled in the art. Derivatives may also be derived from insertion, deletion or substitution of amino acids. Derivatives also include fragments having particular parts of the protein fused to peptides, polypeptides or other proteinaceous or non-proteinaceous molecules. For example, fibulin-1 may be fused to a molecule to facilitate its localisation to the airway tissue.

As used herein a “variant” of fibulin-1 means a molecule which exhibits at least some of the functional activity of the form of fibulin-1 of which it is a variant. A variant may take any form and may be naturally or non-naturally occurring.

As used herein a “homologue” means that the molecule is derived from a species other than that which is being treated in accordance with the method of the present invention. This may occur, for example, where it is determined that a species other than that which is being treated produces a form of fibulin-1 which exhibits similar and suitable functional characteristics to that of the fibulin-1 which is naturally produced by the subject undergoing treatment.

### Diagnosis

The determination that increased fibulin-1 levels are found in the airway tissue, serum and bronchoalveolar lavage fluid of asthmatic individuals provides a means of screening individuals to determine whether the individual suffers from or is otherwise susceptible or predisposed to conditions associated with airway inflammation and/or airway tissue remodelling, such as asthma.

Accordingly, an aspect of the invention provides a method for diagnosing a condition associated with airway inflammation and/or airway tissue remodelling, or susceptibility or predisposition thereto, in a subject, the method comprising determining the level of fibulin-1 in a biological sample from the subject.

It should be understood that the biological sample which is screened in accordance with this aspect of the present invention may be any suitable sample which would be indicative of the fibulin-1 level of the airway tissue. The sample may be a biopsy sample of the airway tissue or it may be some other form of sample such as blood (serum), induced sputum, exhaled breath condensate, or lavage sample.

Although the preferred method is to screen for an increase in fibulin-1 levels in order to diagnose susceptibility to a condition, the detection of a decrease in the level of this molecule may be desired under certain circumstances, for example, to monitor for improvement or responsiveness to a therapeutic or prophylactic treatment regimen. The present invention should therefore be understood to extend to screening for decreases in fibulin-1 levels. The person of skill in the art would appreciate that in the context of this type of screening protocol one may seek to analyse a screening result relative to an earlier obtained result rather than only relative to normal levels.

Methods of screening for levels of fibulin-1 can be achieved by any suitable method which would be well known to persons of skill in the art. In this regard, it should be understood that reference to screening for the level of protein and/or gene expression “in a subject” is intended as a reference to the use of any suitable technique which will provide information in relation to the level of expression of fibulin-1 in the relevant tissue of the subject. Accordingly, these screening techniques include both in vivo screening techniques, as well as in vitro techniques which are applied to a biological sample extracted from the subject. Such in vitro techniques are likely to be preferred due to their significantly more simplistic and routine nature.

Since embodiments of the present invention are predicated, in part, on screening for changes in the level of fibulin-1 such changes can in fact be screened for at the protein level or at the nucleic acid level, such as by screening for increases in the level of fibulin-1 mRNA transcripts. The person of skill in the art will determine the most appropriate means of analysis in any given situation. However it is generally preferred that screening be performed in the context of protein molecules due to the relative simplicity of the techniques which are likely to be utilised. Nevertheless in certain situations, and in the context of particular biological samples, it may be desirable or otherwise useful to directly analyse gene transcription.

Without limiting the scope of the invention in any way, suitable methods of identification of fibulin-1 levels include in vivo molecular imaging (e.g. Weissleder, R et al., Nature Medicine, 6:351-355, 2000), fluorescent in situ hybridisation (FISH), quantitiative reverse transcriptase PCR (QRTPCR), flow cytometry, and immunoassay such as enzyme-linked immunosorbent assay (ELISA). Suitable immunoassay techniques are described, for example, in U.S. Pat. Nos. 4,016,043, 4,424,279 and 4,018,653. These include both single-site and two-site assays of the non-competitive types, as well as traditional competitive binding assays. These assays also include direct binding of a labelled antigen-binding molecule to a target antigen.

Techniques and protocols for employing such methods are well known to those skilled in the art. Any suitable technique may be utilised to detect fibulin-1 or its encoding nucleic acid molecule. The nature of the technique which is selected for use will largely determine the type of biological sample which is required for analysis. Such determinations are well within the scope of the person of skill in the art. Typical samples which one may seek to analyse are biopsy samples of the airways.

The present invention also provides kits suitable for use in accordance with the methods of the invention. Such kits include for example diagnostic kits for assaying biological samples, comprising an agent for detecting fibulin-1 or encoding nucleic acid molecules, and reagents useful for facilitating the detection by the agent(s). Further means may also be included, for example, to receive a biological sample. The agent(s) may be any suitable detecting molecule. Kits according to the present invention may also include other components required to conduct the methods of the present invention, such as buffers and/or diluents. The kits typically include containers for housing the various components and instructions for using the kit components in the methods of the present invention.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### Microarray Analysis of Gene Expression in Airway Smooth Muscle Cells

An analysis of gene expression in airway smooth muscle (ASM) cells of asthmatic and non-asthmatic human subjects was carried out using an Affymetrix human genome microarray. In particular a comparison was made between gene expression in unstimulated ASM cells and ASM cells stimulated with either foetal bovine serum (FBS) (pro-proliferative), interleukin-1β (IL1β) (pro-inflammatory) or transforming growth factor-β (TGFβ)(pro-fibrotic).

ASM cells were isolated from six participants that were age and sex matched (four male and two female), three not with asthma (mean age 23.33 years) and three with asthma (mean age 23 years). Asthmatic subjects were assessed as having, in one case mild/atopic asthma and in the remaining two cases moderate/atopic asthma. ASM cells were isolated from bronchial tissue obtained from either bronchoscopy, lung tissue obtained from lung transplants, or lung tissue resected at thoracotomy by microdissection. Ethical approval for the use of the lung tissue for in vitro experimentation was granted by the Human Ethics Committee of The University of Sydney, and the South Western Sydney Area Health Service, and informed consent was received from all subjects.

This isolation and culture of ASM cells was carried out according to a method described previously by Johnson et al., 1995; 2001. Briefly, with the use of a dissecting microscope, sterile equipment and aseptic technique, bronchial airways were dissected from the surrounding parenchyma and cut longitudinally. The bronchus was then briefly dipped in 70% (v/v) ethanol in water to kill any surface organisms. The bronchus was then washed in sterile Hanks balanced salt solution three times. The bronchus was then pinned down in a sterile Petri dish with the epithelial surface facing upwards. Using a dissecting microscope, the epithelium was removed with fine forceps in order to expose the smooth muscle bundles. The smooth muscle bundles were dissected free from the surrounding tissue and placed in a sterile 15 ml Falcon tube containing Hanks. The Falcon tube was then centrifuged at 200 g for 5 minutes. Isolated pieces of muscle were placed into 25 cm² vented tissue culture flasks (Falcon Labware) containing 2.5 mls of 10% FBS in Dulbecco's modified Eagle's medium (DMEM) supplemented with 20 U/ml penicillin, 2 μg/ml streptomycin, and 250 ng/ml amphotericin B. Flasks were then placed in a humidified CO₂ incubator (5% CO₂ in air) and maintained at 37° C.

ASM cells (passage 7) were seeded in 75 cm² flasks at a density of 1×10⁴ cells/cm² in 5% foetal bovine serum (FBS) (JRH biosciences, Brooklyn, Australia), 1% antibiotics-antimycotic (1 U/ml penicillin, 1 μg/ml streptomycin, and 125 ng/ml amphotericin B) (Invitrogen, Heidelberg, Australia), 1% GlutaMAX™-I supplement (Invitrogen, Heidelberg, Australia) in Dulbecco's modified eagle medium (DMEM) (Invitrogen, Heidelberg, Australia) (growth medium) and grown at 37° C./5% CO₂ in a humidified incubator for nine days. Cells were quiesced in 0.1% bovine serum albumin (BSA) (Sigma Aldrich, St Louis, Mo.), 1% antibiotics-antimycotic, 1% GlutaMAX™-I supplement, DMEM (quiescing medium) for three days, whereafter ASM cells from one flask were washed in cold phosphate buffered saline (PBS) (Invitrogen, Heidlberg, Australia) twice, and stored on ice to be lysed for ribonucleic acid (RNA) extraction using lysis buffer provided in the NucleoSpin RNA II kit (Macherey-Nagel GmbH & co, Duren, Germany) and stored at −20° C. prior to extraction. The remaining flasks were treated with one of the following: quiescing medium, 5% FBS in quiescing medium, 10 ng/mL interleukin-1β (IL-1β)(Pierce Endogen, IL, USA) in quiescing medium or 10 ng/mL transforming growth factor β(TGFβ) (R&D systems, MN, USA) in quiescing medium, and similarly, were lysed for RNA extraction after eight hours of treatment.

Total cellular RNA was isolated using the NucleoSpin RNA II kit (Macherey-Nagel GmbH & co, Duren, Germany) and QIAGEN Total RNA isolation kit (Qiagen, Doncaster, Australia) with a modified protocol as follows. Cells were lysed using 7 μL β-mercaptoethanol (Sigma Aldrich, St Louis, Mo.) and 700 μL RA1 from the NucleoSpin RNA kit. RNA was isolated according to the manufacturer's instructions from the QIAGEN kit, except RNA was eluted in 50 μL of RNase free water. The integrity and purity of RNA was quantified using a Agilent Technologies' 2100 Bioanalyzer (Agilent Technologies, Forest Hill, Australia) and a spectrophotometer respectively. Integrity of the RNA was indicated by the 28S/18S ratio, where a ratio of 1.7 or above indicated non-degraded RNA. RNA samples were stored at −80° C.

5 μg of RNA was reverse transcribed into complimentary deoxyribonucleic acid (cDNA) and further transcribed into cRNA as specified in the manufacturer's instructions (Affymetrix, Calif., USA). Quality of the cRNA was tested using the Agilent Bioanalyser (as above), to indicate no degradation of the RNA sample used. cRNA was hybridised onto the Affymetrix HU-133 Plus 2.0 array according to the manufacturer's instructions (Affymetrix, CA, USA). After 16 hours of hybridisation on the microarray chip, the sample was removed and the chip washed using the Affymetrix® GeneChip® Fluidics Station 400 (Affymetrix, CA, USA) and scanned using the Affymetrix® Scanner (Affymetrix, CA, USA).

Data produced from the microarrays were analysed using Agilent Genespring GX (Version 7.3.1) (Agilent Technologies, Forest Hill, Australia), gene expression of ASM cells isolated from participants with and without asthma following stimulation using: (i) quiescent medium (ii) 5% FBS, (iii) 10 ng/mL IL1β or (iv) 10 ng/mL TGFβ were compared. Genes with more than 10 fold significant upregulation in the asthmatic group compared to the non asthmatic group were considered as possible targets.

Using GeneSpring GX Version 7.3.1, bioinformatic analysis was undertaken. From the 54675 probes on the microarrays, stimulation with 5% FBS, 10 ng/mL IL1 β, or 10 ng/mL TGFβ induced 4340 genes by 10 fold or more. Further, 667 of these genes were significantly upregulated (p<0.05). From this dataset, 76 of the genes were shown to be upregulated in the asthmatic volunteers group compared to the non-asthmatic volunteers.

Upon further analysis, it was identified that one of the genes differentially expressed between asthmatic and non-asthmatic derived ASM cells was fibulin-1. 10 ng/mL TGFβ upregulated fibulin-1 in ASM cells from asthmatic volunteers (24.11 fold increase±15.85), and in contrast, down-regulated fibulin-1 in non-asthmatic volunteers (7.69 fold decrease±0.04).

### Example 2

### Real Time PCR Analysis of Fibulin-1 mRNA in ASM Cells

To verify whether 5% FBS or 10 ng/mL TGFβ upregulated mRNA encoding fibulin-1, and its isoforms, in ASM cells derived from both asthmatic and non-asthmatic volunteers, real time PCR was used. Four isoforms of fibulin-1 (A, B, C and D) have been identified in humans to date. Expression of each of these four isoforms was investigated.

ASM cells derived from 12 non-asthmatic and eight asthmatic volunteers (isolated as described in Example 1), were seeded in six well plates at a density of 1×10⁴ cells/cm² in DMEM containing 5% (v/v) FBS, 1% (v/v) antibiotic-antimycotic (1 U/ml penicillin, 1 μg/ml streptomycin, and 125 ng/ml amphotericin B), and 1% (v/v) glutaMAX I™ (growth medium) and grown at 37° C./5% CO₂ for nine days. Cells were quiesced in 0.1% (w/v) bovine serum albumin (BSA) in 1% (v/v) antibiotic-antimycotic and 1% (v/v) glutaMAX I™ (quiescing medium) for three days, where after, ASM cells from two wells were washed in cold phosphate buffered saline (PBS) twice, and stored on ice and lysed for RNA extraction using lysis buffer provided in the NucleoSpin RNA II kit (Macherey-Nagel GmbH & Co, Duren, Germany) and stored at −20° C. prior to extraction. The remaining wells were treated with one of the following: quiescing medium, 5% FBS in quiescing medium, or 10 ng/mL TGFβ (R&D systems, MN, USA) in quiescing medium, and similarly, were lysed for RNA after eight hours of treatment.

Total cellular RNA was extracted using the NucleoSpin RNA II kit according to the manufacturer's instructions. After extraction, samples were eluted in 50 μl RNase free water and stored at −20° C. until use. 5 μL of RNA was converted into cDNA using RevertAid™ H Minus M-MLV Reverse Transcriptase (Fermentas Life Sciences, Glen Burnie, Md.), with recombinant ribonuclease inhibitor (Fermentas Life Sciences, Glen Burnie, Md.) and dNTPs (Invitrogen, CA, USA) in the presence of random primers (New England Biolabs, MA, USA), according to the manufacturer's instructions.

Real time quantitative PCR was performed in triplicate using the ABI Prism 7000 Sequence Detection System (Applied Biosystems, Foster City, USA). Each 25 μl reaction consisted of 3 μL of the above described cDNA, 12.5 μL of 2× TaqMan Universal Master Mix, No AmpErase UNG Reagents Kit, and 1.25 μL of the 20× predeveloped assay on demand primer (FBLN1: Hs00242545_m1; FBLN1A: Custom Made; FBLN1B: Hs00972625_m1; FBLN1C: Hs00242546_m1; FBLN1D: Hs00197774_m1) and an endogenous eukaryotic control 18S ribosomal RNA primer (all from Applied Biosystems, Foster City, USA). The thermal cycle conditions consisted of 40 cycles of 95° C. for 15 seconds and 60° C. for 1 minute. Data files were generated using 7000 System Software, v1.2.3 (Applied Biosystems, Foster City, USA).

The number of cycles needed to attain a threshold fluorescence (TF) set to lie on the exponential part of the amplification plot (C_{T}) was determined (FBLN1 TF=0.447; FBLN1A TF=0.446; FBLN1 B TF=0.322; FBLN1C TF=0.248; FBLN1D TF=0.302). Results for the common region and isotypes of fibulin-1 were normalised against those obtained for 18S rRNA. Results from triplicate wells from each individual sample were averaged, such that an overall mean and standard error were calculated for cycle number. The data were expressed as a fold change to unstimulated cells.

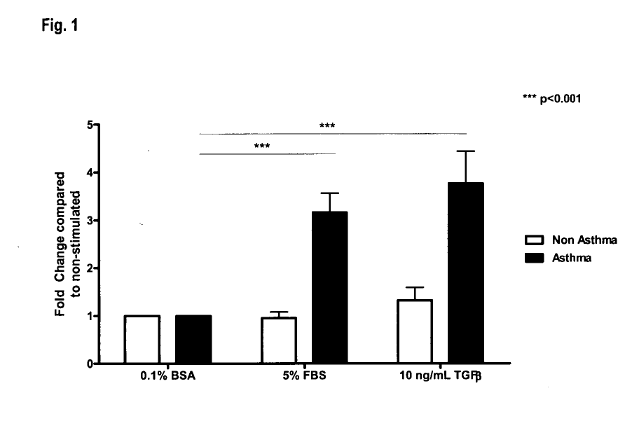

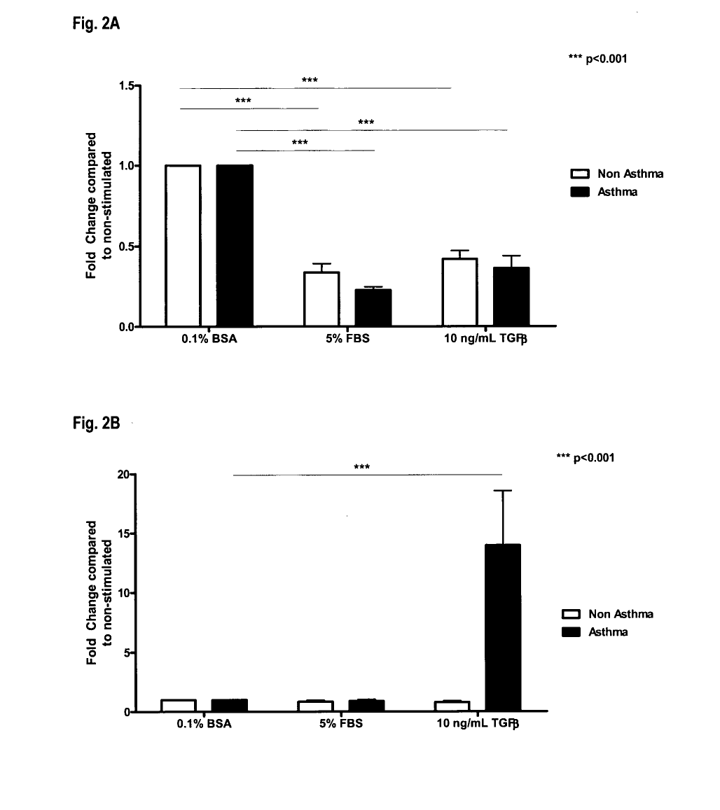

5% FBS and 10 ng/mL TGFβ significantly upregulated mRNA encoding the common region of the four known human fibulin-1 isoforms (3.16±0.13 and 3.77±0.22 fold increase respectively, p<0.001 compared to unstimulated in both cases, two way ANOVA) in ASM cells derived from asthmatics (FIG. 1). This increase was not observed in cells from non-asthmatic volunteers. Further analysis was conducted on each of the four fibulin-1 isoforms, fibulin-1A, fibulin-1 B, fibulin-1C and fibulin-1 D.

### Fibulin-1A

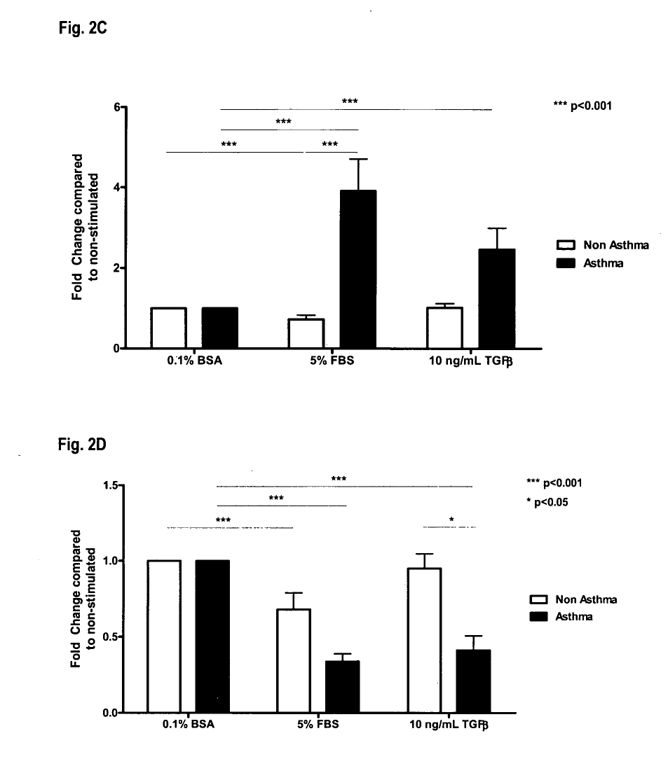

5% FBS and 10 ng/mL TGFβ significantly reduced fibulin-1A in ASM cells derived from both asthmatic (4.37±0.01, 2.76±0.03 fold decrease, p<0.001, p<0.001 respectively compared to unstimulated, two way ANOVA) and non-asthmatic (2.96±0.02, 2.39±0.01 fold decrease, p<0.001, p<0.001 respectively compared to unstimulated, two way ANOVA) volunteers (FIG. 2A).

### Fibulin-1B

10 ng/mL TGFβ significantly upregulated fibulin-1B in ASM cells only derived from asthmatic (13.95±4.60 fold increase, p<0.001 compared to unstimulated, two way ANOVA) volunteers (FIG. 2B). This increase was not observed in cells from non-asthmatic volunteers.

### Fibulin-1C

5% FBS significantly upregulated fibulin-1C in ASM cells from asthmatic volunteers (3.91±0.80 fold increase, p<0.001 compared to unstimulated, two way ANOVA), whilst, downregulated fibulin-1C in non-asthmatic volunteers (1.37±0.14 fold decrease, p<0.001 compared to unstimulated, two way ANOVA). Furthermore, the expression of fibulin-1C was greater in ASM cells from asthmatic compared to non asthmatic volunteers (p<0.001, two way ANOVA) (FIG. 2C). 10 ng/mL TGFβ significantly upregulated fibulin-1C only in ASM cells from asthmatic (2.46±0.53 fold increase, p<0.001 compared to unstimulated, two way ANOVA) volunteers (FIG. 2C).

### Fibulin-1D

5% FBS significantly downregulated fibulin-1D in ASM cells from asthmatic (2.97±0.05 fold decrease, p<0.001 compared to unstimulated, two way ANOVA) and non-asthmatic (1.47±0.11 fold decrease, p<0.01 compared to unstimulated, two way ANOVA) volunteers (FIG. 2D), although the reduction was greater in asthmatics. 10 ng/mL TGFβ significantly downregulated fibulin-1D only in ASM cells from asthmatic volunteers (2.43±0.10 fold decrease, p<0.001 compared to unstimulated, two way ANOVA) (FIG. 2D). Furthermore, the expression of fibulin-1D was lower in ASM cells from asthmatic compared to non asthmatic volunteers (p<0.05, two way ANOVA) (FIG. 2D).

### Example 3

### Western Blot Analysis of Fibulin-1 Expression in ASM Cells

Using western blotting, the change in fibulin-1 protein was measured in ASM cells. The amount of fibulin-1 protein in ASM cells stimulated using 0.1% (w/v) bovine serum albumin (BSA), 5% (v/v) foetal bovine serum (FBS), or 10 ng/mL transforming growth factor β (TGFβ) for 24 hours from asthmatics and non-asthmatics was compared.

ASM cells derived from four non-asthmatic and four asthmatic volunteers were seeded in six well plates at a density of 1×10⁴ cells/cm² in DMEM containing 5% (v/v) FBS, 1% antibiotic-antimycotic (1 U/ml penicillin, 1 μg/ml streptomycin, and 125 ng/ml amphotericin B), and 1% (v/v) glutaMAX I™ (growth medium) and grown for nine days at 37° C./5% 002. Cells were quiesced in 0.1% (w/v) BSA in 1% (v/v) antibiotic-antimycotic and 1% (v/v) glutaMAX I™ (quiescing medium) for three days, where after, ASM cells from two wells were washed in cold phosphate buffered saline (PBS) twice, and stored on ice and lysed for protein using 50 μL of buffer (150 mM NaCl, 50 mM Tris HCl (pH 7.6), 1 mM EDTA, 1% (v/v) Triton X-100, 0.1% SDS, 0.5% deoxycholic acid, 2 mM phenylmethylsulphonyl fluoride (PMSF) and 1% (v/v) protease inhibitor cocktail (Calbiochem, CA, USA)) per well and stored at −20° C. The remaining wells were treated with one of the following: quiescing medium, 5% FBS in quiescing medium, or 10 ng/mL TGFβ (R&D systems, MN, USA) in quiescing medium, and similarly, were lysed for protein extraction after 24 hours of treatment.

Western blot analysis was performed as described previously (Johnson et al., 2006). Briefly, prior to use, samples were centrifuged for 15 minutes (13,000 rpm), mixed with 0.0625 M Tris-HCl (pH 6.8), 2% sodium dodecyl sulfate (SDS), 0.1M 1,4-Dithio-DL-threitol (DTT), 10% glycerol, 0.01% bromophenol blue (loading buffer) in a ratio of 1:5 and denatured at 95° C. for 5 minutes. Samples were loaded and separated on a 10% polyacrylamide gel. 25 μg of positive control (SK-BR-3, human breast carcinoma cell line) (Abgent, CA, USA) was also loaded. Polyvinylidene fluoride (PVDF) (Immobilon-P, 0.45 μm pore size) (Millipore, MA, USA) was cut to size and pre wet using 100% methanol for 10 minutes and titrated into transfer buffer (25 mM Tris-HCl (pH 8.5), 192 mM glycine (pH 8.5), 10% methanol) for 15 minutes. Once the protein was transferred onto the membrane, it was blocked using 5% (w/v) BSA in PBS with 0.05% (v/v) Tween-20 (PBS-Tw) at 4° C. overnight on an orbital shaker. The membrane was incubated with a monoclonal mouse anti-human fibulin-1 (10 μg in 10 mls of 1% (w/v) BSA PBS-Tw) antibody (Alexis Biochemicals, CA, USA) for one hour at room temperature. It was then washed three times with PBS-Tw and a biotinylated goat anti-mouse antibody (Millipore, MA, USA) was added at a 1:20 000 dilution in 1% (w/v) BSA PBS-Tw, and incubated for one hour at room temperature. The membrane was washed three times with PBS-Tw and streptavidin horseradish peroxidise (HRP) (5 μg in 10 mls 1% (w/v) BSA PBS-Tw) (R&D Systems, MN, USA) was added for one hour at room temperature. After washing with PBS-Tw the membrane was visualised following the addition of photoluminescent substrate (West Dura Extended, Pierce Biotechnology, IL, USA) and the image was captured and densitometry calculated using a Kodak image station IS4000 MM.

The membrane was reprobed with a monoclonal mouse anti-human glyceraldehyde-3-phosphate dehydrogenase (GAPDH) (0.5 μg in 10 mls of 1% (w/v) BSA PBS-Tw) antibody (Millipore, MA, USA) for two hours at room temperature. It was then washed three times with PBS-Tw and a goat anti-mouse IgG HRP (4 μg in 10 mls of 1% (w/v) BSA PBS-Tw)(Santa Cruz Biotechnology, CA, USA) was added for one hour at room temperature. After washing with PBS-Tw the membrane was visualised and imaged as described above. Densitometric data was analysed in Kodak MI software (version 4.0.5). Fibulin-1 densitometric values were normalised against those obtained for GAPDH on the same membrane and expressed as a fold change to non stimulated cells. In addition, levels of fibulin-1 were normalised against the positive control, SK-BR-3 whole cell lysates.

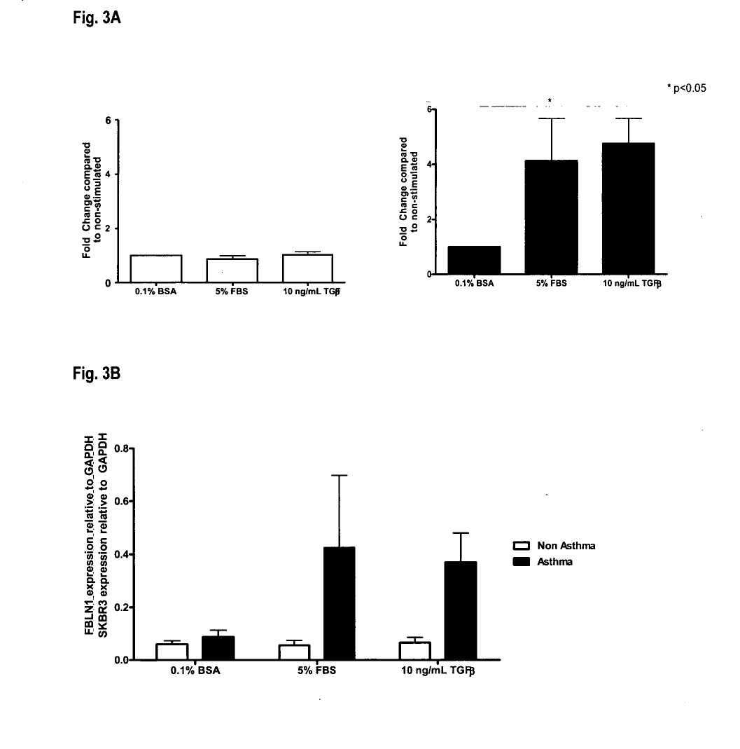

Fibulin-1 protein was detected in ASM cells from both the four asthmatic and four non-asthmatic volunteers. As shown in FIG. 3A, 10 ng/mL of TGFβ significantly upregulated fibulin-1 protein only in ASM cells from asthmatic volunteers (4.75±0.91 fold increase, p<0.05, one way ANOVA). The basal expression of fibulin-1 protein was not significantly different between ASM cells derived from asthmatic and non-asthmatic cells (0.09±0.03 normalised densitometric units (ndu), 0.06±0.01 ndu respectively, p>0.05, unpaired Student's t-test) (FIG. 3B).

### Example 4

### Immunohistochemical Analysis of Fibulin-1 Expression in ASM Cells and Airway Tissue

ASM cells from a total of four asthmatic and five non-asthmatic volunteers, airway sections from seven non-asthmatic volunteers and bronchial biopsy specimens from three asthmatic and three non-asthmatic volunteers were examined using immunohistochemistry.

For cell staining, ASM cells derived from five non-asthmatic and four asthmatic volunteers were seeded in eight well chamber slides (Nalge Nunc, NY, USA) at a density of 1×10⁴ cells/cm² in DMEM containing 5% (v/v) foetal bovine serum (FBS), 1% antibiotic-antimycotic (1 U/ml penicillin, 1 μg/ml streptomycin, and 125 ng/ml amphotericin B), and 1% (v/v) glutaMAX I™ (growth medium) and grown for nine days at 37° C./5% 002. Cells were quiesced in 0.1% (w/v) bovine serum albumin (BSA) in 1% (v/v) antibiotic-antimycotic and 1% (v/v) glutaMAX I™ (quiescing medium) for three days, where after, ASM cells were treated with one of the following: quiescing medium, 5% FBS in quiescing medium, or 10 ng/mL TGFβ (R&D systems, MN, USA) in quiescing medium. After 24 hours stimulation, cells were fixed using cold 4% paraformaldehyde for 20 minutes at 4° C.

For airway rings, a piece of human bronchus was dissected from harvested lung samples of seven non-asthmatic volunteers and cut transversely into rings. One ring per well was placed in a 24 well plate and stimulated with 1 mL per well of one of the following: quiescing medium, 5% FBS in quiescing medium, or 10 ng/mL TGFβ in quiescing medium for 24 hours. The ring was embedded in optimal cutting temperature (OCT) compound and frozen at −80° C.

For airway biopsies, the biopsy was obtained from the bronchial airways of three non-asthmatic and three asthmatic volunteers by endobronchial bronchoscopy of the right middle lobe. The biopsy was placed in a 48 well plate and stimulated with 500 μL of either quiescing medium, 1 ng/mL TGFβ in quiescing medium, or 5 ng/mL TGFβ in quiescing medium for 24 hours. The sample was embedded in OCT compound and frozen at −80° C.

Airway rings and biopsies were cut into 7 μm sections using a cryostat and mounted onto glass slides (Menzel GmbH & Co KG, Braunschweig, Germany). Sections were fixed using 75% (v/v) ethanol, 25% (v/v) acetone for 10 minutes, followed by air drying for 5 minutes. Slides were stored at −80° C. prior to staining.

On the day of staining, the slides were left at room temperature for 5 minutes before rehydrating in water for 10 minutes. The slides were then put into phosphate buffered saline (PBS) for an additional 10 minutes. Samples were blocked in 10% (v/v) non-immune horse serum in PBS for 30 minutes to one hour at room temperature and washed in PBS three times. For cells, 100 μl/well, and for sections, 50 μl/sample of either a monoclonal mouse anti-human fibulin-1 antibody (Alexis Biochemicals, CA, USA) diluted at 2 μg/ml with PBS or a mouse IgM isotype control (Millipore, Boronia, Australia) diluted to 2 μg/ml in PBS was added to each well or section and incubated for one hour at room temperature. After incubation, slides were washed in PBS three times. For cells, 100 μl/well, and for sections, 50 μl/sample of either a monoclonal mouse anti-human fibulin-1 antibody (Alexis Biochemicals, CA, USA) diluted at 1:100 with PBS or a mouse IgM isotype control (Millipore, Boronia, Australia) diluted to the same concentration in PBS as the anti-human fibulin-1 antibody was added to each well or section and incubated for one hour at room temperature. After incubation, slides were washed in PBS three times. The primary antibody for fibulin-1 and the isotype control antibody were detected by the addition of either a texas red (Vector Laboratories, CA, USA) or fluorescein isothiocyanate (FITC) (BD Biosciences, North Ryde, Australia) conjugated anti-mouse antibody, diluted 1:100 in PBS for 30 minutes at room temperature in the dark. Slides were washed in PBS three times prior to mounting using Vectashield® mounting media (Vector Laboratories, CA, USA). Images were taken on an Olympus fluorescence microscope (model BX51) and captured using Leica IM1000 image capture software. The fluorescent threshold was set using the corresponding isotype control. Using this same threshold, staining specific for fibulin-1 or fibronectin was measured. A sequential section was stained with haematoxylin and eosin to enable orientation of the sample.

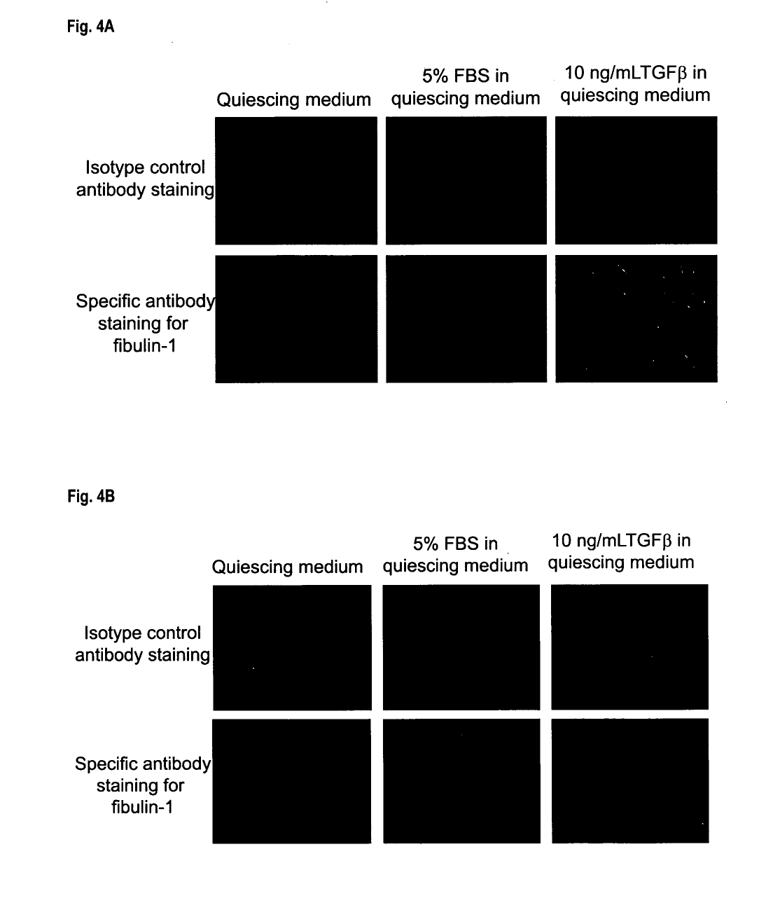

As shown in FIG. 4A, 10 ng/mL TGFβ increased the intensity of staining for fibulin-1 in ASM cells from asthmatic volunteers. This observation was consistent in all four asthmatic ASM cells studied. In comparison, and as shown in FIG. 4B, the intensity of staining for fibulin-1 remained constant in the presence of either stimulus in ASM cells derived from five non-asthmatic volunteers.

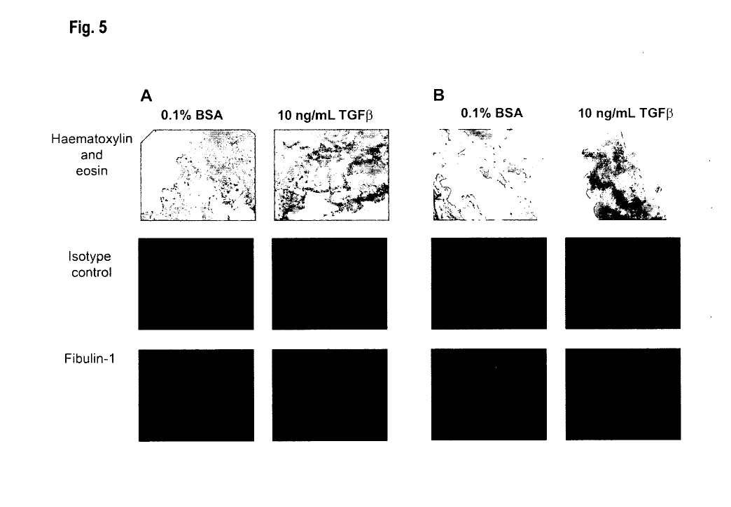

To confirm the expression of fibulin-1 in bronchial ring tissue, sections of bronchial ring tissue were taken from seven non-asthmatic volunteers. However, as shown in FIG. 5A, positive and consistent staining for fibulin-1 was observed in all bronchial ring tissue sections and this was not modulated by any of the stimuli.

As shown in FIG. 5B, TGFβ (10 ng/mL) increased the intensity of fibulin-1 staining in bronchial biopsy sections derived only from the three asthmatic volunteers studied.

### Example 5

### Colocalisation and Interaction of Fibulin-1 and Fibronectin in Airway Tissue

It is known that fibulin-1 interacts with fibronectin. The present inventors therefore investigated whether these two proteins co-localised in cells and tissue of the airways. ASM cells from four asthmatic and five non-asthmatic volunteers, and airway sections from seven non-asthmatic volunteers were examined using FRET and immunohistochemistry respectively.

ASM cells were seeded in eight well chamber slides (Nalge Nunc, NY, USA) at a density of 1×10⁴ cells/cm² in DMEM containing 5% (v/v) FBS, 1% antibiotic-antimycotic (1 U/ml penicillin, 1 μg/ml streptomycin, and 125 ng/ml amphotericin B), and 1% (v/v) glutaMAX I™ (growth medium) and grown for nine days at 37° C./5% 002. Cells were quiesced in 0.1% (w/v) bovine serum albumin (BSA) in DMEM with 1% (v/v) antibiotic-antimycotic and 1% (v/v) glutaMAX I™ (quiescing medium) for three days, where after, ASM cells were treated with one of the following: quiescing medium, 5% FBS in quiescing medium, or 10 ng/mL transforming growth factor β (TGFβ) in quiescing medium. After 24 hours stimulation, cells were fixed using cold 4% paraformaldehyde for 20 minutes at 4° C.

For airway rings, a piece of human bronchus was dissected from the harvested lung sample and cut transversely into rings. One ring per well was placed in a 24 well plate and stimulated with 1 mL per well of one of the following: quiescing medium, 5% FBS in quiescing medium, or 10 ng/mL TGFβ in quiescing medium for 24 hours. The ring was embedded in optimal cutting temperature (OCT) compound and frozen at −80° C. Airway rings were cut into 7 μm sections using a cryostat and mounted onto glass slides (Menzel GmbH & Co KG, Braunschweig, Germany). Sections were fixed using 75% (v/v) ethanol, 25% (v/v) acetone for 10 minutes, followed by air drying for 5 minutes. Slides were stored at −80° C. prior to staining.

For ASM cells, samples were blocked in 1% (w/v) BSA in PBS for 30 minutes at room temperature and washed in PBS 3 times. A Zenon Rabbit IgG Labelling Kit was used to label the antibodies with Alexa Fluor 555 and Alexa Fluor 647 (Molecular Probes, Heidelberg, Australia). Polyclonal rabbit anti-human fibulin-1 antibody (4 μg/mL in PBS) (Santa Cruz Biotechnology, Santa Cruz, Calif., USA) and polyclonal rabbit anti-human fibronectin antibody (3 μg/mL in PBS) were conjugated with Alexa Fluor 555 (12 μg/mL in PBS) and Alexa Fluor 647 (1 μg/mL in PBS) in 10 μL PBS respectively. After 5 minutes incubation in the dark, to block any non-conjugated Alexa Fluor fragments, non-specific rabbit IgG (Alexa Fluor 555: 300 μg/mL and Alexa Fluor 647: 25 μg/mL, both in PBS) was added to each sample and incubated in the dark for a further 5 minutes. The two antibody samples were combined together in PBS (antibody:zenon complex). One hundred μL of the antibody:zenon complex was added to each well of the chamber slide and incubated for 1.5 hours at room temperature in the dark. After incubation, slides were washed in PBS 3 times and refixed in cold 4% paraformaldehyde for 20 minutes at 4° C. Slides were washed in PBS 3 times prior to being coverslipped using 15% (v/v) glycerol in PBS, pH 8.0.

For airway rings, samples were blocked in 10% (v/v) non-immune horse serum in phosphate buffered saline (PBS) for 30 minutes to one hour at room temperature and washed in PBS three times. 50 μL/sample of either a monoclonal mouse anti-human fibulin-1 antibody (Alexis Biochemicals, CA, USA) diluted at 2 μg/mL in PBS and a polyclonal rabbit anti-human fibronectin antibody (Sigma Aldrich, MO, USA) diluted at 1.2 μg/mL in PBS, or a mouse IgM isotype control (Millipore, Boronia, Australia) diluted at 2 μg/mL and a rabbit IgG isotype control (Dako, Botany, Australia) diluted at 1.2 μg/mL, was added to each section and incubated for one hour at room temperature. After incubation, slides were washed in PBS three times. The primary antibody for fibulin-1 and the corresponding isotype control antibody were detected using a goat anti-mouse fluorescein isothiocyanate (FITC) conjugated antibody (BD Biosciences, North Ryde, Australia), diluted at 1 μg/mL in PBS and incubated for 30 minutes at room temperature in the dark. Fibronectin and its corresponding isotype control were detected using a goat anti-rabbit Tetramethyl Rhodamine Iso-Thiocyanate (TRITC) conjugated antibody (Sigma Aldrich, MO, USA), diluted at 6.67 μg/mL in PBS and incubated for 30 minutes at room temperature in the dark. Slides were washed in PBS three times prior to being mounted using 15% (v/v) glycerol in PBS. Images were taken on an Olympus fluorescence microscope (model BX51) and captured using Leica IM1000 image capture software. The fluorescent threshold was set using the corresponding isotype control. Using this same threshold, staining specific for fibulin-1 or fibronectin was measured. Images were superimposed using Adobe Photoshop CS3 (Adobe Systems, Chatswood, Australia).

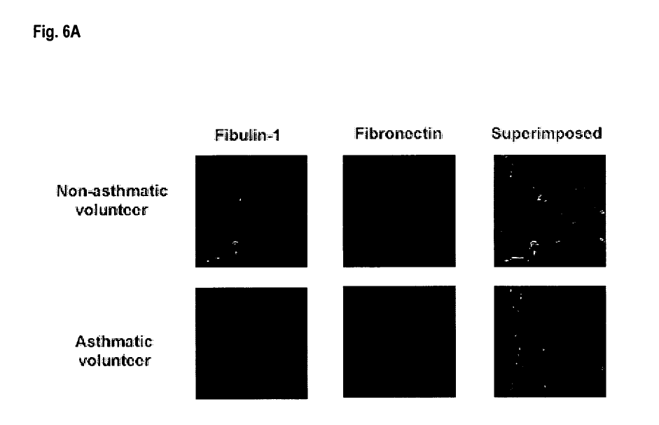

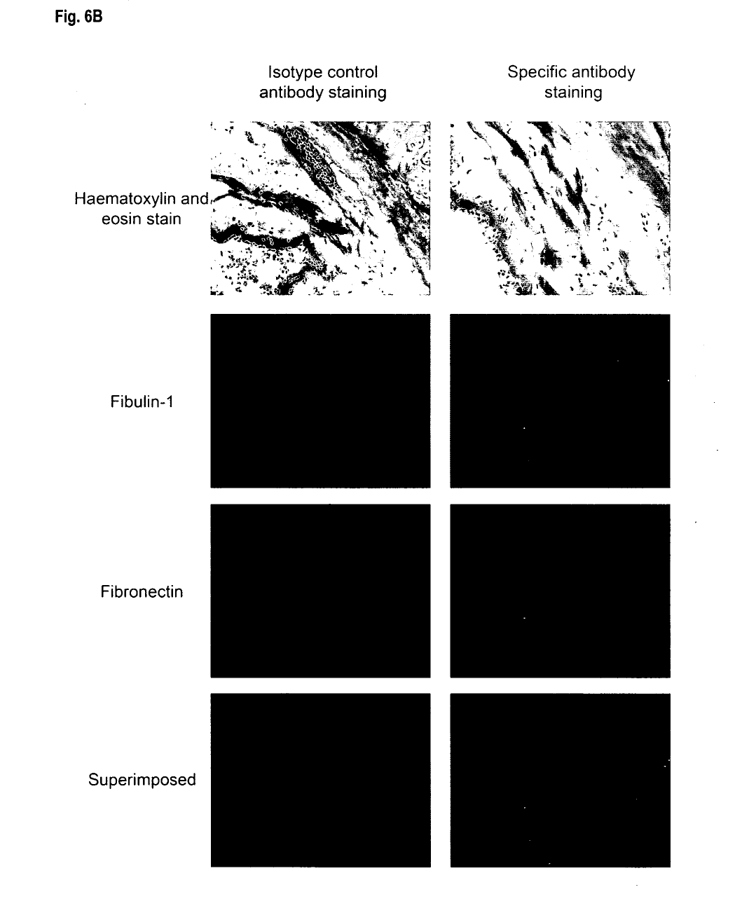

As shown in FIG. 6A, fibulin-1 and fibronectin are colocalised in the same region of the ASM cells from both asthmatic and non-asthmatic volunteers. This observation was consistent in the ASM cells from four asthmatic and five non-asthmatic volunteers. To confirm that colocalisation of fibulin-1 and fibronectin is also observed in bronchial ring tissue, sections of bronchial ring tissue were taken from seven non-asthmatic volunteers and immunohistochemically examined. As shown in FIG. 6B, staining for fibulin-1 and fibronectin was present in bronchial ring tissue sections from the seven non-asthmatic volunteers in comparison to the isotype control staining. Fibulin-1 and fibronectin were found to be colocalised in the same region of the tissue section, in particular, in the basement membrane and airway smooth muscle bulk.

Fluorescence resonance energy transfer (FRET) approaches were used to demonstrate the physical interaction between fibulin-1 and fibronectin in ASM cells from both asthmatic and non-asthmatic volunteers (data not shown).

### Example 6

### Deposition of Fibulin-1 and Fibronectin into the Extracellular Matrix of ASM Cells

Fibulin-1 is an extracellular protein. The inventors therefore investigated whether fibulin-1 is deposited into the extracellular matrix (ECM) formed by airway smooth muscle (ASM) cells from asthmatic and non-asthmatic volunteers. The deposition of fibulin-1 into the ECM was measured using an enzyme linked immunosorbent assay (ELISA). In addition, in view of the interaction between fibulin-1 and fibronectin, the inventors also measured the deposition of fibronectin into the ECM using an ELISA.

ASM cells derived from ten non-asthmatic and ten asthmatic volunteers were seeded in 96 well plates at a density of 1×10⁴ cells/cm² in DMEM containing 5% (v/v) FBS, 1% antibiotic-antimycotic (1 U/ml penicillin, 1 μg/ml streptomycin, and 125 ng/ml amphotericin B), and 1% (v/v) glutaMAX I™ (growth medium) and grown for nine days at 37° C./5% 002. Cells were quiesced in 0.1% (w/v) bovine serum albumin (BSA) in DMEM with 1% (v/v) antibiotic-antimycotic and 1% (v/v) glutaMAX I™ (quiescing medium) for three days, and treated with one of the following: quiescing medium, or 10 ng/mL TGFβ in quiescing medium. After 24 hours stimulation, cells were washed five times in phosphate buffered solution with 0.05% Tween-20 (PBS-Tw), excess solution removed, and lysed using 0.016 mM NH₄OH. After 20 minutes at 37° C., the wells were washed five times with PBS-Tw and stored with 150 μL of PBS-Tw per well at −20° C. until analysed.

One day prior to the assay, the 96 well plate was thawed, washed an additional five times and blocked using 200 μL of 1% (w/v) BSA PBS-Tw per well overnight at 4° C. On the day of the assay, the plate was washed five times using PBS-Tw. Analysis was performed in triplicate, using 50 μL/well of either a monoclonal mouse anti-human fibulin-1 antibody (Alexis Biochemicals, CA, USA) diluted 2 μg/mL with 1% (w/v) BSA PBS-Tw, a monoclonal mouse anti-human fibronectin antibody (Millipore, Boronia, Australia) diluted at 1.2 μg/mL with 1% (w/v) BSA PBS-Tw, a mouse IgM isotype control (Millipore, Boronia, Australia) at 2 μg/mL with 1% (w/v) BSA PBS-Tw, or an anti-mouse IgG₁ isotype control (BD Biosciences, North Ryde, Australia) diluted at 1.2 μg/mL with 1% (w/v) BSA PBS-Tw per well. The plate was incubated for two hours at room temperature with orbital shaking before being washed five times using PBS-Tw. In wells measuring fibulin-1 or its isotype control, 50 μL/well of a biotinylated sheep anti-mouse antibody (Millipore, Boronia, Australia) diluted 5 μg/mL with 1% (w/v) BSA PBS-Tw was added to each well. In wells measuring fibronectin or its isotype control, 50 μL/well of PBS-Tw was added to each well. The plate was incubated for one hour at room temperature with orbital shaking before washing five times using PBS-Tw. For wells measuring fibulin-1 or its isotype control, 50 μL/well of streptavidin (conjugated with HRP) (R&D systems, MN, USA) diluted 0.5 μg/mL with 1% (w/v) BSA PBS-Tw was added to each well. For wells measuring fibronectin or its isotype control, 50 μL/well of a rabbit anti-mouse antibody (conjugated with HRP) (Dako, Botany, Australia) diluted at 0.5 μg/mL with 1% (w/v) BSA PBS-Tw was added to each well. The plate was incubated for 45 minutes at room temperature with orbital shaking. The plate was then washed five times and developed using 50 μL/well of 2-2′-azino-bis(3-ethylbenzthiazoline-6-sulfonic acid) liquid substrate (ABTS) (Sigma Aldrich, St Louis, Mo.) on an orbital shaker until colour developed. The reaction was stopped using 1 mM phosphoric acid (Sigma Aldrich, St Louis, Mo.) and quantified by reading the plate at 405 nm.

Results from triplicate wells were averaged, and the mean absorbance value for the isotype control was subtracted from the mean absorbance value for the specific antibody for each sample. The data were expressed as a fold change compared to non stimulated cells.

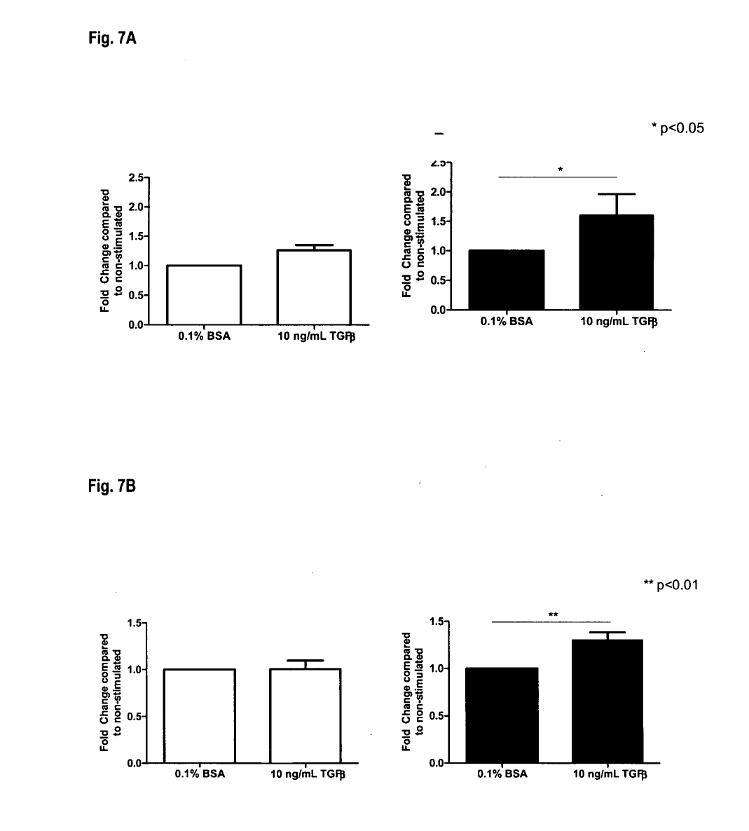

10 ng/mL TGFβ significantly upregulated fibulin-1 deposition in the ECM from ASM cells of 10 asthmatic volunteers (1.60±0.12 fold increase, p<0.05 compared to unstimulated, paired Student's t-test), however, this increase was not observed in the 10 non-asthmatic volunteers studied (1.26±0.03 fold increase, p>0.05 compared to unstimulated, paired Student's t-test) (FIG. 7A).

Similarly, 10 ng/mL TGFβ significantly upregulated fibronectin deposition into the ECM from ASM cells of 10 asthmatic volunteers (1.30±0.03 fold increase, p<0.01 compared to unstimulated, paired Student's t-test). This was not observed in the 9 non-asthmatic volunteers studied (1.01±0.03 fold increase, p>0.05 compared to unstimulated, paired Student's t-test) (FIG. 7B).

### Example 7

### Fibulin-1 Levels in ASM Cell Supernatant, Serum and Bronchoalveolar Lavage Fluid

The release of soluble fibulin-1 from ASM cells from asthmatic and non-asthmatic volunteers was measured using dot blotting. Specifically, using specialised dot blotting equipment (Slot Blot manifold, PR 648, GE healthcare) fibulin-1 in supernatants from ASM cells from asthmatic and non-asthmatic volunteers stimulated using 0.1% (w/v) bovine serum albumin (BSA), 5% (v/v) foetal bovine serum (FBS), or 10 ng/mL transforming growth factor β (TGFβ) for 24 hours was measured. In addition, the amount of fibulin-1 in the serum, and BALF of asthmatic and non-asthmatic volunteers was compared.

Volunteers with intermittent, mild persistent, or moderate persistent atopic asthma (according to the guidelines of the Global Initiative for Asthma, 2005, Global Strategy for Asthma Management and Prevention) were recruited by advertisement (aged between 19 and 30 years). Inclusion criteria included asthma symptoms in the preceding 12 months, a positive mannitol bronchial provocation challenge test, and fewer than 5 pack year history of smoking. Asthma symptoms persisted for at least 8 years in all volunteers with asthma. Volunteers who had a baseline forced expiratory volume predicted in 1 second (FEV₁) of less than 60% predicted, had taken inhaled or systemic corticosteroids within the preceding 2 months, had a history of respiratory failure or intubation, had smoked in the preceding 12 months or had symptoms of an upper respiratory tract infection were excluded.

Normal healthy volunteers with no history of asthma, or other lung disease were recruited by advertisement (aged between 19 and 33 years). Inclusion criteria included a normal baseline spirometry (at least 80% predicted), negative mannitol bronchial challenge test, and no significant bronchodilator reversibility (12% and 200 mL improvement in FEV₁).

Approval of the study was provided by the Ethics Review Committee of the South West Sydney Area Health Service, Royal Prince Alfred Hospital Zone and The University of Sydney human ethics committee.

Samples of bronchoalveolar lavage fluid (BALF) and serum were taken from volunteers both with and without asthma. Samples were taken from volunteers with asthma before and after inhaled corticosteroid treatment for 7 weeks. All volunteers with asthma received inhaled fluticasone propionate (either 100 μg or 500 μg twice daily) via a metered dose aerosol (Glaxo Smith Kline, GSK) and large volume spacer device (Volumatic spacer, GSK). Volunteers were also provided with salbutamol (metered dose aerosol, 100 mcg, Ventolin, GSK), a short acting β2 agonist, for use on an as needed basis for the duration of the study to relieve symptoms of asthma including dyspnoea, chest tightness, wheeze and cough. BALF samples from volunteers with and without asthma were taken by the instillation of 0.9% sodium chloride solution (4 successive aliquots of 60 mL, total of 240 mL), which was warmed to 37° C., into the right middle lobe. It was aspirated for collection in a sterile glass bottle. BALF was filtered through sterile gauze and centrifuged at 2000 rpm for 5 minutes. Fifty millilitres of the acellular supernatant was removed and stored at −70° C.

Polyvinylidene fluoride (PVDF) (Immobilon-P, 0.45 μm pore size) (Millipore, MA, USA) was cut to size and pre wet using 100% methanol for 10 minutes and titrated into phosphate buffered saline with 0.05% Tween (PBS-Tw) for 15 minutes before placing in the blot machine. 50 μL of either human serum (diluted 1:1000 in PBS-Tw), BALF (no dilution), supernatant (no dilution), foetal bovine serum (diluted 1:2 in PBS-Tw) (positive control), or PBS-Tw (negative control) was loaded into each slot and vacuum applied until the samples had gone through. The membrane was washed three times with PBS-Tw and the flow through removed with the aid of the vacuum before removal of the membrane from the apparatus. The membrane was blocked in 5% (w/v) BSA for 1 hour at room temperature. Primary antibody, mouse anti-human fibulin-1 (Alexis Biochemicals, CA, USA), was added at a 1 μg/mL dilution in 1% (v/w) BSA PBS-Tw and incubated for one hour at room temperature. The membrane was then washed three times with PBS-Tw and a biotinylated goat anti-mouse secondary antibody (Millipore, MA, USA) added at a 1:20 000 dilution in 1% (w/v) BSA PBS-Tw, and incubated for one hour at room temperature. After washing three times with PBS-Tw, streptavidin horseradish peroxidise (HRP) (R&D Systems, MN, USA) was added at a 0.5 μg/mL dilution in 1% (w/v) BSA PBS-Tw for one hour at room temperature. The membrane was washed with PBS-Tw for 15 minutes and visualised following the addition of photoluminescent substrate (West Dura Extended, Pierce Biotechnology, IL, USA). The image was captured and densitometry values calculated using a Kodak image station IS4000 MM and Kodak MI (version 4.0.5) software. Densitometric analysis of fibulin-1 was normalised by subtracting the value shown in the negative control (PBS alone) and expressed as a fold change compared to the positive control, 50% FBS.

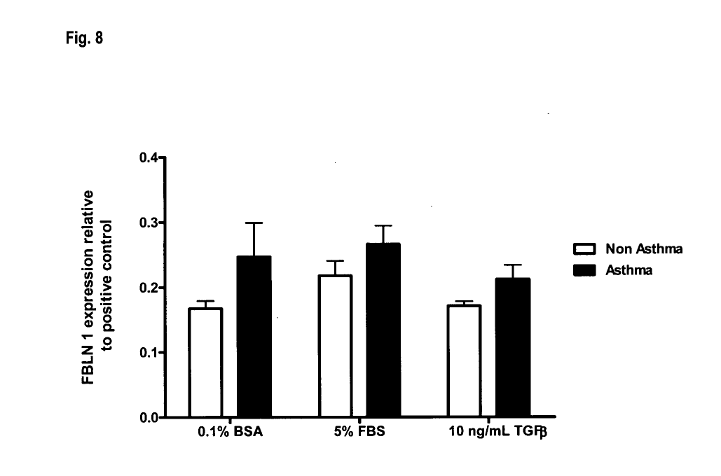

Fibulin-1 protein was detected in the supernatants from ASM cells derived from five asthmatic and four non-asthmatic volunteers (FIG. 8). No significant differences were found between the concentration of fibulin-1 in the supernatant produced from ASM cells from asthmatic versus non asthmatic volunteers stimulated with 0.1% (w/v) BSA (0.25±0.05 normalised densitometric units (ndu), n=5; 0.16±0.01 ndu, n=4 respectively, p=0.23); 5% (v/v) FBS (0.27±0.03 ndu, n=5; 0.22±0.02 ndu, n=4 respectively, p=0.24); or 10 ng/mL TGFβ (0.21±0.02 ndu, n=5; 0.17±0.01 ndu, n=4 respectively, p=0.15).

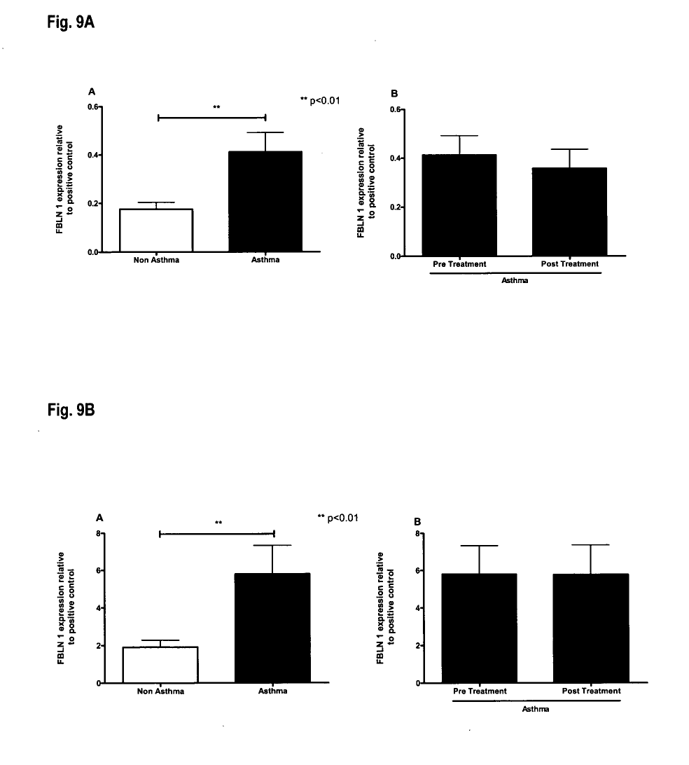

As shown in FIG. 9A, increased fibulin-1 levels were found in serum from asthmatic volunteers who were not being treated with corticosteroids (0.41±0.08 ndu, n=21) versus non-asthmatic volunteers (0.18±0.03 ndu, n=20, p=0.009, Student's unpaired t-test). Corticosteroids did not have any effect on fibulin-1 levels in serum from asthmatic volunteers. As shown in FIG. 9B, increased fibulin-1 levels were found in the BALF from asthmatic volunteers who were not being treated with corticosteroids (6.0±1.6 ndu, n=20) versus non-asthmatic volunteers (1.9±0.37 ndu, n=11 p=0.006, Student's unpaired t-test). Corticosteroids did not have any effect on fibulin-1 levels in BALF from asthmatic volunteers.

### Example 8

### Fibulin-1 Inhibits the Development of Airway Hyperresponsiveness in a Murine Model of Asthma

To investigate the effect of inhibiting fibulin-1 on asthma pathology, antisense oligonucleotides specific for fibulin-1C were designed (Professor Steve Wilton, University of Western Australia). Sequences of two fibulin-1 C specific antisense oligonucleotides (JSR1306 and JSR1307) and one scrambled oligonucleotide are shown below.

JSR1306:
(SEQ ID NO: 9)
5′-gca agc gcu cac agc ggc ugc aag-3′
JSR1307:
(SEQ ID NO: 10)
5′-cuu gcu aag acu uua uua acg cc-3′
scrambled:
(SEQ ID NO: 11)
5′-auu uug ucu gaa acc cug uaa aga g-3′

Successful transfection of the antisense oligonucleotides into ASM cells was achieved using a commercially available kit (Qiagen, Doncaster, Australia). To verify the successful incorporation of the antisense oligonucleotides into ASM cells, and their ability to silence fibulin-1C, quantitative real time polymerase chain reaction (PCR) and western blotting were used (as described above).

In the absence of stimulation, JSR1306 significantly decreased mRNA encoding fibulin-1C (1.50±0.19 fold decrease, p<0.05 compared to effectene only, two way ANOVA) in ASM cells derived from non-asthmatics (data not shown). JSR1307 significantly decreased mRNA encoding fibulin-1C in ASM cells derived from both non-asthmatic and asthmatic volunteers (1.82±0.11, 1.73±0.05 fold decrease, p<0.01 and p<0.05 compared to effectene only respectively, two way ANOVA). 10 ng/mL TGFβ significantly upregulated fibulin-1C in ASM cells from asthmatic volunteers transfected with either effectene only or the scrambled antisense oligonucleotide (1.55±0.15, 1.72±0.27 fold increase respectively, p<0.05 and p<0.01 compared to unstimulated, two way ANOVA). In contrast, in the presence of 10 ng/mL TGFβ stimulation JSR1306 and JSR1307 significantly reduced mRNA encoding fibulin-1C in ASM cells derived from asthmatic volunteers (1.56±0.13, 1.51±0.05 fold decrease, p<0.01 compared to TGFβ stimulated and transfected with effectene only, two way ANOVA) (data not shown).

The inventors then investigated the role of fibulin-1 in airway hyperresponsiveness (AHR) in a murine model of asthma and determined if an inhibitor of fibulin-1, an antisense oligonucleotide, can reverse AHR in this murine model. As mice only contain a single fibulin-1 isoform (whereas the human genome encodes four isoforms) a novel antisense olignucleotide mixture (AO) was designed for the murine studies. For the studies described below, each of the following three antisense oligonucleotides were used in equal amounts in the AO:

17A/1497:
(SEQ ID NO: 12)
5′-cac uug gcg cac gac acc ugg gga-3′
15D/1498:
(SEQ ID NO: 13)
5′-uga acu uga ucc acu cac ccu cag g-3′
15A/1499:
(SEQ ID NO: 14)
5′-uuu ucu cuu ggc gga agc ugc aga-3′

Adult Balb/c mice (6-8 weeks old) were administered 200 ng of recombinant human TGFβ (R&D systems) on days 0 and 1 intranasally (IN) in PBS. Briefly, mice were warmed briefly using a heat lamp and then were anesthetised by IV injection with 12.5 mg/kg Alfaxalone (Jurox, NSW, Australia). Mice were then suspended vertically and were orotracheally intubated with a 22-gauge flexible plastic catheter (Terumo Sureflo, Hospital Supplies of Australia). Endotracheal positioning was confirmed by palpation of the tracheal rings with the catheter tip. Mice were administered 200 ng TGFβ in 40 ul PBS or 40 ul of PBS alone on day 0 and day 1. Animals remained vertical for 1-2 min after administration to ensure the inoculums remained in the lungs.

Mice in both the asthma model (TGF) and the control groups were administered the AO against fibulin or a scrambled AO (fibulin AO control) intranasally (IN). Briefly, mice were anesthetised with isofluorane (4%) and treated with either 40 ug AO or 40 ug scrambled oligonucleotide in 40 ul of PBS, or 40 ul of PBS alone by intranasal droplet application. Treatment occurred daily for the first 7 days and then 3 times a week for three weeks.

AHR was assessed in vivo by measuring changes in transpulmonary resistance and dynamic compliance using a Buxco (Buxco, Sharon, USA) mouse plethysmograph on day 29. Mice were anaesthetised with an intraperitoneal injection of ketamine/xylazine (80-100 mg/kg and 10 mg/kg, respectively) and cannulated via the trachea with an 18G metal tube. Mice were then mechanically ventilated (150 strokes/min, 175 ul stroke volume). Volume changes due to thoracic expansion with ventilation were measured by a transducer connected to the plethysmograph flow chamber. Once stabilized, mice were challenged with saline, followed by increasing concentrations of methacholine (0.625, 1.25, 2.5, 5, 10 and 20 mg/ml), aerosolized by an ultrasonic nebuliser and administered (10 μl) directly to the lungs via the inspiratory line. Each aerosol was delivered for a period of 5 min, during which pressure and flow data were continuously recorded, and a computer program (BioSystemXA; Buxco Electronics) was used to calculate pulmonary resistance and compliance. Peak values were taken as the maximum response to the concentration of methacholine being tested, and were expressed as the percentage change over the saline control. Statistical analysis was performed by a two-way ANOVA using Graphpad Prism 5 software. Mice were then euthanized by sodium pentobarbital overdose and tissue collected as described below.

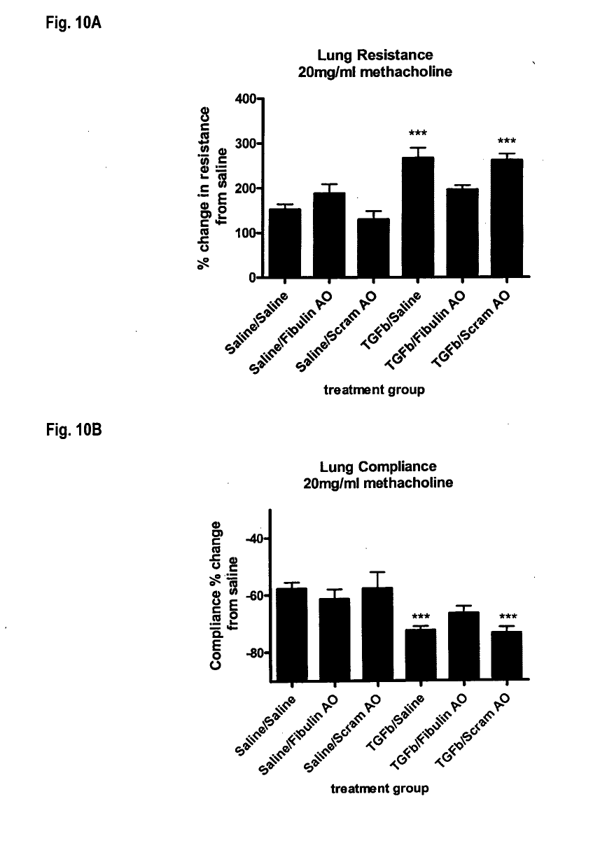

As can be seen in FIG. 10A, the mice which received treatment with TGFβ alone demonstrated an increase in specific airway resistance to methacholine in comparison to naïve (saline) treated animals demonstrating that the inventors had successfully established a murine model of asthma (p<0.05, n=6 Saline, n=7 TGFβ methacholine (20 mg/ml) one way ANOVA with Dunnett's post test). The mice which had received concurrent treatment with Fibulin AO and TGFβ had no alteration in specific airway resistance to methacholine (20 mg/ml) in comparison to naïve animals (p>0.05, n=6 Saline, n=7 fibulin AO+TGFβ for each, one way ANOVA with Dunnett's post test). Moreover, the mice which had received concurrent treatment with a scrambled fibulin AO and TGFβ had increased specific airway resistance to methacholine (20 mg/ml) in comparison to naïve animals (p<0.01, n=6 Saline, n=7 scrambled AO+TGFβ, one way ANOVA with Dunnett's post test). In the absence of TGFβ, mice treated with fibulin AO or scrambled AO had no alteration in their specific airway resistance to methacholine.

Similarly, as can be seen in FIG. 10B, the mice which received treatment with TGFβ alone had decreased specific airway compliance to methacholine in comparison to naïve (saline) treated animals (p<0.01, n=6 Saline, n=7 TGFβ 20 ng/ml methacholine (20 mg/ml) one way ANOVA with Dunnett's post test). The mice which had received concurrent treatment with Fibulin AO and TGFβ had no alteration in specific airway compliance to methacholine (20 mg/ml) in comparison to naïve animals (p>0.05, n=6 Saline, n=7 fibulin AO+TGFβ for each, one way ANOVA with Dunnett's post test). Moreover, the mice which had received concurrent treatment with a scrambled fibulin AO and TGFβ had decreased specific airway compliance to methacholine (20 mg/ml) in comparison to naïve animals (p<0.01, n=6 Saline, n=7 scrambled AO+TGFβ, one way ANOVA with Dunnett's post test). In the absence of TGFβ, mice treated with fibulin AO or scrambled AO had no alteration in their decreased specific airway compliance to methacholine.

### Example 9

### Fibulin-1 Promotes Wound Repair in Airway Smooth Muscle Cells of Asthmatic Patients

To investigate a possible functional role of fibulin-1, rates of ASM cell wound healing were examined using a wound healing protocol as follows.

### ASM Cells on an Autologous Extracellular Matrix

ASM cells were grown in 24 well plates at a density of 1×10⁴ cells/cm² for 1 day in 5% FBS, 1% GlutaMAX™-I supplement, supplemented with 1 U/ml penicillin, 1 μg/ml streptomycin, and 125 ng/ml amphotericin B (1% antibiotics) in Dulbecco's Modified Eagle's Medium (DMEM) (Invitrogen, Carlsbad, Calif., USA) (Growth medium) before being transfected using the protocol described in Example 8. ASM cells were treated with no antisense or Effectene® only, or transfected with 200 nM scrambled antisense oligonucleotide or the antisense oligonucleotide JSR1307, for 3 days. ASM cells were then quiesced in quiescing medium for 3 days. A wound, approximately 1 cm in length, was induced in the cell monolayer with a yellow pipette tip (QSP, Pathtech, Preston, Australia). The wells were washed twice with sterile PBS and replenished with either quiescing medium or 10 ng/mL TGFβ in quiescing medium for up to 72 hours.

### ASM Cells on a Non-Autologous Extracellular Matrix

To observe the effect of fibulin-1 in the ECM, ASM cells were seeded in 75 cm² flasks at a density of 1×10⁴ cells/cm² in growth medium at 37° C./5% CO₂. After 24 hours, ASM cells either underwent medium change (growth medium) or were transfected using JSR1307 in growth medium for a further 3 days using the protocol described in Example 8. The cells were then trypsinised and reseeded in 24 well plates at a density of 1×10⁴ cells/cm² in growth medium, maintaining the presence or absence of JSR1307. Cells were left to grow for 4 days, following which the cells were lysed using 0.016 mM sterile NI-140H. After 20 minutes at 37° C., the wells were washed 5 times with sterile PBS and stored at −20° C. with 500 μL of sterile PBS per well.

On the day of seeding, the pre-laid ECM was defrosted and washed once in DMEM. ASM cells were seeded at a density of 1×10⁴ cells/cm² in growth medium at 37° C./5% CO₂. After 24 hours growth, the cells were treated with growth medium alone or Effectene® only, or transfected with 200 nM scrambled antisense oligonucleotide or JSR1307, using the protocol described in Example 8 for 3 days. ASM cells were then quiesced in quiescing medium for 3 days. A wound, approximately 1 cm in length, was induced in the cell monolayer with a yellow pipette tip (QSP, Pathtech, Preston, Australia). The wells were washed twice with sterile PBS and replaced with either quiescing medium or 10 ng/mL TGFβ in quiescing medium.

The wound was observed using phase contrast microscopy on an Olympus microscope (model CK2) and images of the entire wound were taken at 0, 16, 24, 48 and 72 hours post wound induction with an Olympus digital camera (model 0-4000). Images of each wound were merged into a single image, such that the image contained the whole wound using Adobe Photoshop CS3. The area of each wound at each different time point, in pixels, was measured by digitally drawing around the perimeter of the wound using ImageJ 1.37v software. In each of the studies described below initial wound size was determined to be equivalent between treatment groups (data not shown). The percentage of wound closure compared with the initial wound size was calculated using equation:

$$\% wound closure compared with 0 hr = 100 - 100 * \frac{( Wound {area}_{tx} )}{Wound {area}_{t 0}}$$

Where ₜₓ is the wound area at a given time point and ₜ₀ is the initial wound area

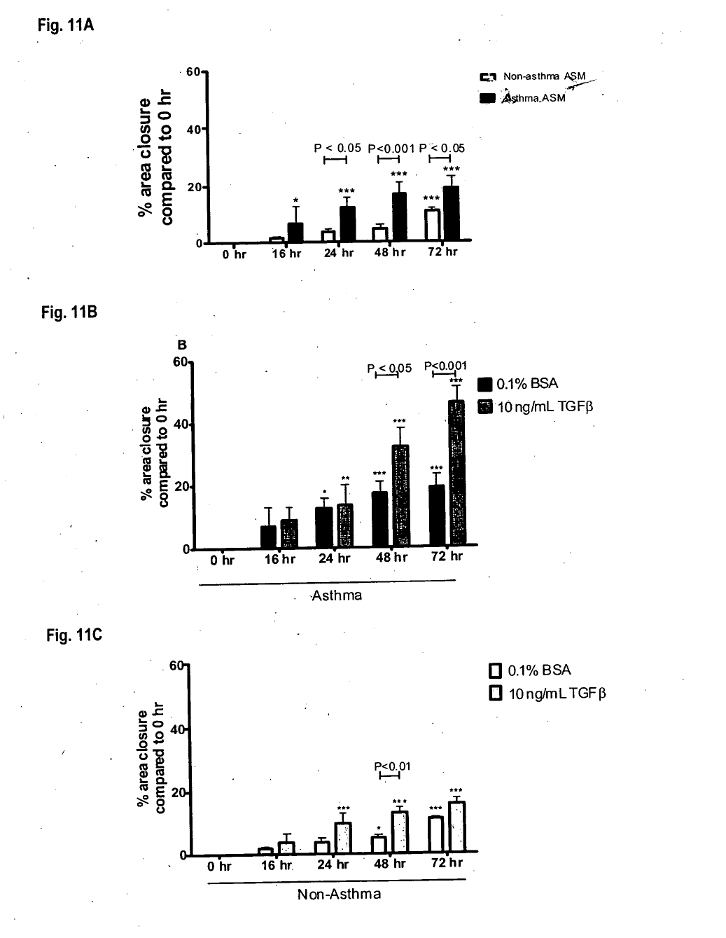

Initially, the differences in wound healing between ASM cells derived from asthmatic and non-asthmatic volunteers were examined. Basally, wound closure of ASM cells derived from asthmatic and non-asthmatic volunteers occurred 16 and 72 hours post wound induction (P<0.001, P<0.05 respectively; two way ANOVA) (FIG. 11A). 24 hours post wound induction, wound closure of ASM cells derived from asthmatic participants was faster than that derived from non-asthmatics (P<0.05; two way ANOVA). 48 hours post wound induction, stimulation using 10 ng/mL TGFβ significantly increased wound healing rates of ASM cells derived from both asthmatics (FIG. 11B) and non-asthmatics (FIG. 11C) (P<0.01, P<0.05 respectively; two way ANOVA).

To further investigate the effects of fibulin-1 on wound healing in ASM cells, the expression of fibulin-1C was silenced using the antisense oligonucleotide JSR1307 (see Example 8). This was compared with the effect of a scrambled antisense oligonucleotide as a control.

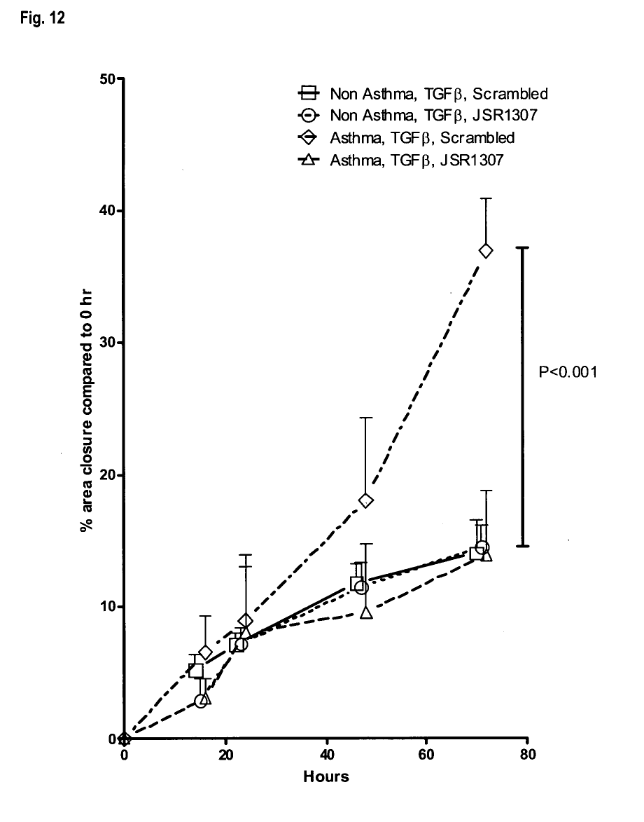

Silencing fibulin-1 did not affect wound healing rates of ASM cells derived from non-asthmatic volunteers (FIG. 12). In the case of ASM cells derived from asthmatics basally, silencing fibulin-1 did not alter wound healing rates whereas following 10 ng/mL TGFβ stimulation silencing fibulin-1 decreased wound healing rates 72 hours post wound (P<0.01; two way ANOVA). As shown in FIG. 12, at 72 hours post wound induction it can be seen that wound healing of ASM cells derived from asthmatic volunteers transfected with scrambled oligonucleotide was faster than in ASM cells derived from non-asthmatics equivalently treated (P<0.001; two way ANOVA). However, when fibulin-1 was silenced in ASM cells derived from asthmatics using JSR1307, the increased wound healing rate was abrogated such that the rate was not different from that of ASM cells derived from non-asthmatics (P>0.05, two way ANOVA).

As the rate of wound healing differed in ASM cells derived from asthmatic and non-asthmatic volunteers, to determine the mechanism underlying the increased wound healing ASM cells were seeded on a non-autologous ECM derived from asthmatic or non-asthmatic volunteers. ASM cells derived from asthmatic volunteers have increased rates of wound healing compared with those derived from non-asthmatics. This was further confirmed in ASM cells seeded on asthma-derived non-autologous ECM in the absence and presence of 10 ng/mL TGFβ (data not shown). Basally, wound closure of ASM cells, derived from both asthmatics and non-asthmatics, seeded onto asthma derived ECM (n=1) occurred 24 hours post wound induction (P<0.001, P<0.05 respectively; two way ANOVA). 72 hours post wound induction, the rate of wound healing of ASM cells derived from asthmatics was greater compared to that of non-asthmatics (P<0.01; two way ANOVA). In the presence of 10 ng/mL TGFβ, significant wound closure of ASM cells, derived from both asthmatics and non-asthmatics) seeded onto asthma derived ECM (n=1) occurred 16 hours post wound induction (P<0.01 in both cases; two way ANOVA). 48 hours post wound induction, the rate of wound healing of ASM cells derived from asthmatics was greater compared to that of non-asthmatics (P<0.01; two way ANOVA).

In the absence or presence of TGFβ, 16 hours post wound induction, fibulin-1 silenced ASM cells derived from asthmatics seeded on asthma-derived ECM inhibited wound closure (P<0.05, P<0.001 respectively; two way ANOVA). Interestingly, the same pattern was not observed when asthma derived ASM cells were seeded on non-asthma derived ECM. Silencing of fibulin-1 in ASM cells derived from asthmatics seeded on non-asthma derived ECM did not alter the rate of wound healing in the absence or presence of 10 ng/mL TGFβ (P>0.05 in both cases; two way ANOVA).

Since ASM cells seeded on asthma derived ECM had increased wound healing rates and increased fibulin-1 deposition levels in the ECM, the effect of fibulin-1 in the ECM on wound healing was examined. As fibulin-1 could not be further silenced in non-asthma derived ECM to effectively investigate the effects of fibulin-1 in the ECM, asthma derived ECM was used. In addition, since it was previously demonstrated that Effectene® and scrambled antisense transfection do not alter wound healing rates, these were omitted in subsequent experiments.

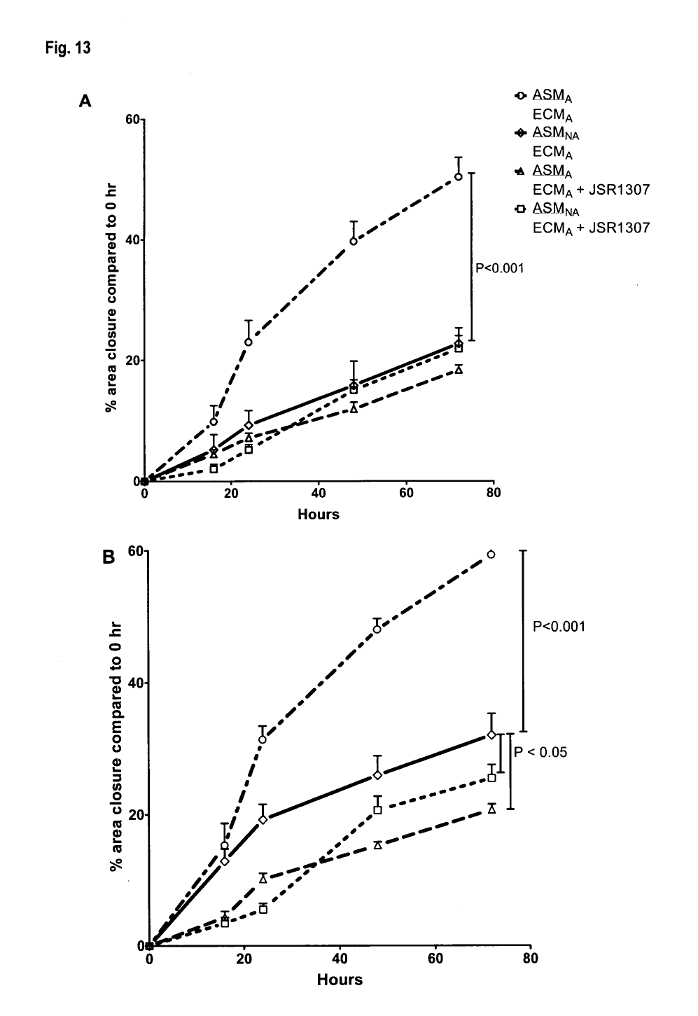

In summary, silencing fibulin-1 in the ECM decreased rates of wound healing in ASM cells derived from asthmatics. As shown in FIG. 13A, basally at 72 hours post wound induction wound healing of ASM cells derived from asthmatics seeded on ECM silenced for fibulin-1 was slower compared with those seeded on ECM where fibulin-1 was present (P<0.001; two way ANOVA). Furthermore, by silencing fibulin-1 in the ECM, the increased wound healing rate was abrogated such that the rate was not different from that of ASM cells derived from non-asthmatics seeded on an ECM where fibulin-1 was present (P>0.05; two way ANOVA). In the presence of 10 ng/mL TGFβ, the same pattern was observed 72 hours post wound induction (P<0.001; two way ANOVA) (FIG. 13B). Furthermore, the rate of wound healing of ASM cells derived from non-asthmatics seeded on ECM silenced for fibulin-1 was also slower compared with those seeded on ECM where fibulin-1 was present (P<0.05; two way ANOVA). By silencing fibulin-1 in the ECM, the increased wound healing rate observed in ASM cells derived from asthmatics was abrogated such that the rate was further decreased compared with that of ASM cells derived from non-asthmatics seeded on an ECM where fibulin-1 was present (P<0.05; two way ANOVA).

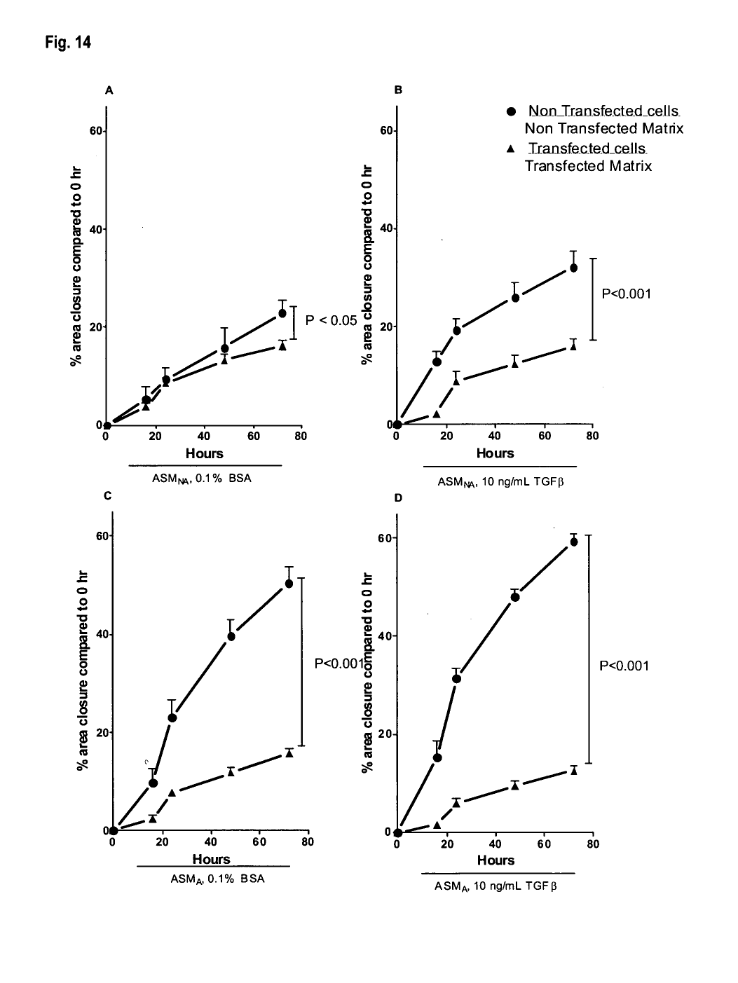

Since it was demonstrated that silencing fibulin-1 in either compartment (ASM cells or ECM deposited with ASM cells) significantly reduced the rate of wound healing, the effect of silencing fibulin-1 in both compartments was examined. To prove that an additive effect of silencing fibulin-1 in both compartments existed, a system whereby fibulin-1 was present in both compartments was compared with a system where fibulin-1 was absent in both compartments. 72 hours post wound induction, silencing of fibulin-1 in both compartments significantly decreased rates of wound healing in ASM cells derived from both asthmatics and non-asthmatics, both in the absence and presence of 10 ng/mL TGFβ (P<0.05 in all cases; two way ANOVA) (FIG. 14).

Further, basally, silencing fibulin-1 in one compartment (either ASM or ECM) did not alter wound healing rates compared with silencing fibulin-1 in both compartments (P>0.05; two way ANOVA) (data not shown). In the presence of 10 ng/mL TGFβ, fibulin-1 silenced ECM reduced rates of wound healing compared with a system where fibulin-1 was present in both ECM and ASM (P<0.001; two way ANOVA). When fibulin-1 was silenced in both compartments wound healing rates were further reduced when compared with fibulin-1 silenced in ECM only (P<0.001; two way ANOVA). However, the rate of wound healing was not different between ASM silenced for fibulin-1 and where fibulin-1 was silenced in both compartments (P>0.05; two way ANOVA). Silencing fibulin-1 in ASM or ECM significantly decreased the rate of wound closure of ASM cells but an additive effect of silencing both ASM and ECM was not observed. Therefore, silencing fibulin-1 in either compartment appears to be sufficient to decrease wound closure.

### REFERENCES

Argraves, W S et al., 1990, Fibulin is an extracellular matrix and plasma glycoprotein with repeated domain structure. J Cell Biol 111:3155-3164.
Johnson, P R et al., 1995, Heparin and PGE2 inhibit DNA synthesis in human airway smooth muscle cells in culture. Am J Physiol 269:L514-519.
Johnson, P R et al., 2001, Airway smooth muscle cell proliferation is increased in asthma. Am J Respir Crit Care Med 164:474-477.
Johnson, P R et al., 2006, Connective tissue growth factor induces extracellular matrix in asthmatic airway smooth muscle. Am J Respir Crit Care Med 173:32-41.
Pupa, S M et al., 2003, Monoclonal antibody to fibulin-1 generated by genetic immunization. J Cell Biochem 89:647-652.

1. A method for treating or preventing a condition associated with airway inflammation and/or airway tissue remodelling, the method comprising administering to a subject in need thereof an effective amount of at least one agent capable of inhibiting the expression and/or activity of fibulin-1.

2. The method of claim 1 wherein the agent is an antagonist of the activity of the fibulin-1 polypeptide.

3. The method of claim 1 wherein the agent is a molecule(s) capable of inhibiting or suppressing expression of fibulin-1.

4. The method of claim 3 wherein the inhibition of expression is at the level of the fibulin-1 nucleotide sequence.

5. The method of claim 4 wherein the agent is an antisense construct capable of specifically binding to a nucleotide sequence encoding fibulin-1 or one or more isoforms thereof.

6. The method of claim 1 wherein the inhibition of the expression and/or activity of fibulin-1 occur in one or more cells and/or in the extracellular matrix associated with such cells.

7. A method for the treatment or prevention of asthma, the method comprising administering to a subject suffering from, or predisposed to, asthma an effective amount of at least one agent capable of inhibiting the expression and/or activity of fibulin-1.

8. A method of regulating the occurrence of airway tissue remodelling in a subject, said method comprising modulating the level of fibulin-1 in airway tissue of the subject, wherein normalising fibulin-1 levels in said tissue relative to normal endogenous levels reduces the occurrence of, or inhibits, tissue remodelling.

9. The method of claim 8 wherein the normalising of fibulin-1 levels relative to normal endogenous levels involves decreasing fibulin-1 by administering an effective amount of one or more agents capable of decreasing the expression or production of fibulin-1.

10. The method of claim 8 wherein the normalising of fibulin-1 levels relative to normal endogenous levels involves increasing fibulin-1 by administering an effective amount of fibulin-1, a derivative, variant or homologue thereof, or an agent capable of increasing the expression or production of fibulin-1.

11. A method for the treatment or prevention of a condition associated with airway inflammation and/or airway tissue remodelling in a subject, the method comprising modulating the level of fibulin-1 in the airway tissue of the subject wherein normalising fibulin-1 levels in said tissue relative to normal endogenous levels downregulates the occurrence of tissue remodelling.

12. The method of claim 1 wherein the fibulin-1 is the isoform fibulin-1C.

13. The method of claim 1 wherein the condition is selected from asthma, chronic obstructive pulmonary disease (COPD), pulmonary fibrosis including idiopathic pulmonary fibrosis, cystic fibrosis and lymphangioleiomyomatosis (LAM).

14. A method for diagnosing a condition associated with airway inflammation and/or airway tissue remodelling, or susceptibility or predisposition thereto, in a subject, the method comprising determining the level of fibulin-1 in a fluid or airway tissue or cells of the subject.

15. The method of claim 14 wherein the fluid is serum or bronchoalveolar lavage fluid.

16. The method of claim 14 wherein the cells are airway smooth muscle cells.

17. The method of claim 14 comprising isolating fluid or airway cells from the subject and determining the level of expression of at least one fibulin-1 isoform in the fluid or airway cells, wherein the level of expression of at least one fibulin-1 isoform is indicative of the condition, or a susceptibility or predisposition thereto.

18. The method of claim 17 wherein airway cells are isolated and stimulated in vitro using one or more pro-proliferative or pro-fibrotic factors prior to determination of fibulin-1 expression.

19. A pharmaceutical composition for treating or preventing a condition associated with airway inflammation and/or airway tissue remodelling, the composition comprising at least one agent capable of inhibiting the expression and/or activity of fibulin-1 in airway tissue, optionally together with one or more pharmaceutically acceptable adjuvants, carriers and/or diluents.

20. A method for inhibiting aberrant, excessive or otherwise unwanted wound repair in a tissue of a subject, wherein the subject suffers from or is predisposed to a condition associated with airway inflammation and/or airway tissue remodelling, the method comprising administering to the subject an effective amount of at least one agent capable of inhibiting the expression and/or activity of fibulin-1.

21. The method of claim 20 wherein the condition is selected from asthma, chronic obstructive pulmonary disease (COPD), pulmonary fibrosis including idiopathic pulmonary fibrosis, cystic fibrosis and lymphangioleiomyomatosis (LAM).

22. The method of claim 20 wherein the inhibition of wound repair comprises inhibiting the expression and/or activity of fibulin-1 in one or more abnormal cells.

23. The method of claim 22 wherein the cells are airway smooth muscle cells.

24. The method of claim 20 wherein the inhibition of wound repair comprises inhibiting the expression and/or activity of fibulin-1 in the extracellular matrix.
